(19) United States
(12) Patent Application Publication
(10) Pub. No.: US 2009/0054374 A1
(43) Pub. Date: Feb. 26, 2009

(54) METHODS OF TREATING ACUTE EXACERBATIONS OF CHRONIC OBSTRUCTIVE PULMONARY DISEASE

(75) Inventor: Thomas P. Kennedy, Charlotte, NC (US)

Correspondence Address: ALSTON & BIRD LLP, BANK OF AMERICA PLAZA, 101 SOUTH TRYON STREET, SUITE 4000, CHARLOTTE, NC, 28280-4000, US

(73) Assignee: Paringenix, Inc.

(21) Appl. No.: 12/039,137

(22) Filed: Feb. 28, 2008

Related U.S. Application Data
(60) Provisional application No. 60/989,562, filed on Nov. 21, 2007.
(60) Provisional application No. 60/892,053, filed on Feb. 28, 2007.

(51) Int. Cl. A61K 31/727; A61P 11/12

(52) U.S. Cl. 514/56

(57) ABSTRACT

The present invention provides methods for treating and preventing acute exacerbations of Chronic Obstructive Pulmonary Disease. The methods particularly comprise administering to a patient have COPD a composition comprising O-desulfated heparin. The administration can be after onset of one or more symptoms indicating an exacerbation of COPD or prior to onset of such symptoms. After onset of an acute exacerbation, administration of the O-desulfated heparin is particularly beneficial for reducing the time of hospitalization of the patient and for reducing lung inflammation.

### CROSS-REFERENCE TO RELATED APPLICATIONS

The present invention claims priority to U.S. Provisional Patent Application No. 60/989,562, filed Nov. 21, 2007, and U.S. Provisional Patent Application No. 60/892,053, filed Feb. 28, 2007, both of which are incorporated herein by reference in their entirety.

### FIELD OF THE INVENTION

The invention relates to methods of treating and preventing acute exacerbations of pulmonary diseases, and particularly acute exacerbations of chronic obstructive pulmonary disease.

### BACKGROUND

Chronic Obstructive Pulmonary Disease (“COPD”) is an umbrella term used to describe a group of respiratory tract diseases generally characterized by airflow obstruction or limitation. This condition may also be known under the terms chronic obstructive respiratory disease (CORD), chronic obstructive airways disease (COAD), chronic obstructive lung disease (COLD), or chronic airway limitation (CAL). As used herein, the term COPD is intended to encompass all such references.

The Global Initiative for Chronic Obstructive Lung Disease (GOLD) defines COPD as a disease state characterized by airflow limitation that is not fully reversible. The airflow limitation is usually progressive and associated with abnormal inflammatory response of the lungs to noxious particles or gases. The American Thoracic Society (ATS) defines COPD as a disease process involving progressive chronic airflow obstruction because of chronic bronchitis, emphysema, or both. Chronic bronchitis is defined clinically as excessive cough and sputum production on most days for at least three months during at least two consecutive years. Emphysema is characterized by chronic dyspnea (shortness of breath) resulting from the destruction of lung tissue and the enlargement of air spaces. A further condition typically encompassed by the term COPD is bronchiectasis, which is an abnormal stretching and enlarging of the respiratory passages caused by mucus accumulation and blockage. Under such conditions, the weakened passages can become scarred and deformed, allowing more mucus and bacteria to accumulate, resulting in a cycle of infection and blocked airways.

Asthma is an inflammatory disease of lung airways that makes the airways prone to narrow too much and too easily in response to a wide variety of provoking stimuli. Although asthma features airflow obstruction, asthma is not typically encompassed by the term COPD since the pulmonary function deficits of asthma are reversible.

COPD is generally recognized as one of the most serious and disabling conditions in middle-aged and elderly Americans. The main risk factor in the development of COPD is cigarette smoking. It is estimated that approximately 15% of all chronic smokers will develop the disease, and cigarette smoking is implicated in 90% of diagnosed cases of COPD. COPD can also be caused by prolonged exposure to certain dusty environments, such as the coal mining and grain storage industries.

COPD is a progressive, incurable disease wherein chronic inflammation of the cells lining the bronchial tree plays a prominent role (although the exact pathophysiology thereof is still not completely understood). Smoking, and occasionally other inhaled irritants, perpetuates an ongoing inflammatory response that leads to airway narrowing and hyperactivity. As a result, airways become edematous, excess mucus production occurs, and cilia function poorly. With disease progression, patients have increasing difficulty clearing secretions. Consequently, they develop a chronic productive cough, wheezing, and dyspnea. Bacterial colonization of the airways leads to further inflammation and the formation of diverticula in the bronchial tree.

The clinical course of COPD is characterized by chronic disability, with intermittent, acute exacerbations that occur more often during the winter months. An acute exacerbation of COPD can be defined as a sustained worsening of the patient's symptoms from his or her usual stable state that is beyond normal day-to-day variations, and is acute in onset. When acute exacerbations occur, they typically manifest as increased sputum production, more purulent sputum, change in sputum color, increased coughing, upper airway symptoms (e.g., colds and sore throats), increased wheezing, chest tightness, reduced exercise tolerance, increased fatigue, fluid retention, acute confusion, and worsening of dyspnea. Although infectious etiologies account for most exacerbations, exposure to allergens, pollutants, or inhaled irritants may also play a role. Infectious agents known to cause acute exacerbations of COPD include: rhinoviruses, influenza, parainfluenza, coronavirus, adenovirus, respiratory syncytial virus, Chlamydia pneumoniae, Haemophilus influenzae, Streptococcus pneumoniae, Moraxella catarrhalis, Staphylococcus aureus, Mycloplasma pneumoniae, and Pseudomonas aeruginosa. Pollutants known to cause acute exacerbations include nitrogen dioxide, particulates, sulfur dioxide, and ozone. Despite these known causes, the exact cause of exacerbations may be unidentifiable in up to 30% of diagnosed cases of exacerbation of COPD.

Much has been learned about the pathogenesis of COPD since the early descriptions of emphysema in the 19^{th} century. The airway in stable COPD is characterized by an inflammatory response consisting of macrophages and CD8 T lymphocytes in the airway wall (Saetta M, et al., Am J Respir Crit Care Med 163:1304-1309, 2001; Cosio M G, Eur. Respir J24:3-5, 2004) and of polymorphonuclear neutrophils in the airway lumen. During acute exacerbations, though, the pattern of cellular infiltrate changes dramatically. The concentration of cellular elements in bronchoalveolar lavage (BAL) rises over 50-fold compared to subjects with stable COPD (Drost E M, et al., Thorax 60:293-300, 2005) and neutrophils become the dominant cell of inflammation, not only within the airway lumen (Hurst JR, et al., Am J Respir Crit Care Med 173:71-78, 2006), but also within the airway wall (Qiu Y, et al., Am J Respir Crit Care Med 168:968-975, 2003).

A recent study suggests that neutrophilic airway inflammation is dramatically induced in all COPD exacerbations, regardless of whether the etiology of exacerbation is the consequence of bacterial infection, viral infection, combined bacterial and viral infection, or an exacerbation not characterized by definable pathogens (Papi A, et al., Am J Respir Crit Care Med 173: 1114-1121, 2006). In the BAL from patients with acute exacerbations, neutrophils constitute over half of all cellular elements as a percentage of BAL differential counts, compared to only 18% in the BAL fluid of subjects with stable COPD and 5% in the BAL from non-smokers and healthy smokers. The cause of this neutrophilic inflammatory influx is a dramatic increase in neutrophil-stimulating chemokines within BAL and of chemokine-secreting cells within the airway subepithelium induced acutely by viral and/or bacterial infection of the lung. These include both the cysteine-x-cysteine ligands (CXCL5 and CXCL8) and their receptors (CXCR1 and CXCR2).

Chemokine upregulation results in prominent staining for the neutrophil protease human leukocyte elastase (HLE) within the airway subepithelium. This is pathophysiologically significant because of its potential for producing proteolytic airway injury (Nadel J A, Chest 117 (Suppl.):386S-389S, 2000; Kohri K, et al., Am J Physiol Lung Cell Mol Physiol 283:L531-L540, 2002) and also because HLE (Kohri, et al., J Clin Invest 85:682-689, 1990; Sommerhoff C P, et al., J Clin Invest 85:682-689, 1990) and other neutrophil proteases stimulate bronchial mucus hypersecretion and possibly activate airway epithelial epidermal growth factor and Toll (Devaney J M, et al., FEBS Lett 544:129-132, 2003) receptors, stimulating pro-inflammatory signaling cascades.

When chemokines signal neutrophil influx from the vascular space into the lung, the first event in efflux involves changes in neutrophil velocity along the vascular wall. The initial attachment of neutrophils on the vascular endothelial wall is mediated by up to three calcium-dependent lectins called selectins. L-selectin is consitutively expressed by neutrophils, and P- and E-selectin are positioned on the surface of endothelial cells that have been activated by inflammation within the organ in which they reside (Sperandio M, FEBS J 273:4377-4389, 2006). Initially, the decrease in neutrophil rolling is produced by the immediate transport of P-selectin to the endothelial cell surface of endothelial cells activated by inflammatory mediators. This leads to an increase in selectin-dependent neutrophil rolling along the endothelial surface of post-capillary venules.

As neutrophils roll, they activate, flatten, and firmly adhere to the endothelial surface through attachment of CD 18 integrins on the neutrophil surface to the intercellular adhesion molecules ICAM-1 and ICAM-2 constitutively expressed on endothelium (Petri B, et al., FEBS J 273:4399-4406, 2006). Finding an intercellular junction between endothelial cells for diapedesis, the neutrophil then transmigrates from the vascular space to the target tissue where it can similarly adhere to cells of the reperfused organ via ICAMs expressed on the surface of target organ cells. By production of the potent oxidant hypochlorous acid (HOCl) and over 20 different proteases, including human leukocyte elastase (HLE), collagenase and gelatinase, the neutrophil can not only engulf invading microbes but is also capable of causing profound and indiscriminate injury to inflamed tissues.

Acute exacerbations of COPD are accompanied by evidence not only of increased airways inflammation but also of increased systemic inflammation. The most commonly used biomarker of inflammation is C-reactive protein (CRP). CRP bind bacteria, oxidized lipids, and apoptotic cells and facilitates their clearance from the immune system (Mold C, et al., J Immunol 168:6375-6381, 2002; Weiser N J, et al., J Exp Med 187:631-640, 1998; and Chang M-K, et al., Proc Natl Acad Sci USA 99:13043-13048, 2002). Slightly increased CRP levels have been shown to accompany the vascular inflammation of atherosclerosis and to predict increased risk of coronary disease and myocardial infarction (Libby P, et al., Am J Med 116:9 S-16S, 2004). CRP has also recently been shown to associate with increased lung inflammation in stable patients with COPD (Gan W Q, et al., Thorax 59:574-580, 2004; de Torres J P, et al., Eur Respir J 27:902-907, 2006; and Pinto-Plata V M, et al., Thorax 61:23-28, 2006). Elevated CRP has been shown to be a strong and independent predictor of future COPD outcomes such as hospitalization and COPD death in individuals with airway obstruction (Dahl M, et al., Am J Respir Crit Care Med 175:250-255, 2007).

Out of 36 individual plasma biomarkers surveyed in a recent study of 90 COPD patients studied in paired fashion at baseline and during exacerbation, only elevation in CRP proved useful in confirming the presence of COPD exacerbation (Hurst JR, et al., Am J Respir Crit Care Med 174:867-874, 2006). A high CRP concentration two weeks after COPD exacerbation strongly predicts that a patient will suffer a subsequent exacerbation within the next 50 days (Perera W R, et al., Eur Respir J 29:527-534, 2007). In cardiovascular disease, patients with elevated CRP are now being targeted for aggressive treatment of vascular inflammation with agents such as statins to lower CRP levels as an indication of successful therapy of coronary and other vascular inflammation.

Similarly, CRP can be also used to monitor inflammation within the airways as it relates to a decline in lung function. In data on 2,633 randomly selected adults monitored 10 years apart, there is an inverse relationship between CRP and forced expiratory volume in the first second of the spirogram (FEV₁) (Fogarty A W, et al., Thorax 62:515-20, 2007). Thus, elevated CRP can be used as a marker of the degree of airways inflammation in stable COPD, a marker of COPD exacerbation and a marker of improvement in airways inflammation over time. Corticosteroids suppress systemic inflammation in stable COPD measured by suppression of serum CRP levels (Paul Man SF, et al., Proc Am Thorac Soc 2:78-82, 2005). Oral or intravenous corticosteroids are a mainstay of therapy for acute exacerbations of COPD (Niewoehner D E, et al., N Engl J Med 340:1941-1947, 1999; Davies L, et al., Lancet 354:456-460, 1999; and de Jong Y P, et al., Chest, published on-line Jul. 23, 2007; DOI 10.1378/chest.07-0208). Nevertheless, patients suffering COPD exacerbations enjoy a fall in CRP of less than 50% by day 7 from the initiation of treatment, even in subjects responding favorably to currently available standard therapy. In fact, according to the most recent literature available, elevated CRP did not fall at all after corticosteroid administration to subjects hospitalized with COPD exacerbations (Bozinovki S, et al., Am J Respir Crit Care Med 177:269-278, 2008) and remained elevated until 30 days after admission.

Periodic exacerbations of COPD are a major cause of morbidity, mortality, and health care costs in patients with COPD. Patients who suffer exacerbations have a worse quality of life (Seemungal T A, et al., Am J Respir Crit Care Med 157:1418-1422, 1998; Spencer S, et al., Eur Respir J23:698-702, 2004; and Niewoehner D E. Et al., Am J Med 119:S38-S45, 2006) and a more rapid decline in both health status and lung function as measured by FEV₁ (Kanner RE, et al., Am J Respir Crit Care Med 164:358-364, 2001; and Donaldson G C, et al., Thorax 57:847-852, 2002). Admission to the hospital for COPD exacerbation is associated with an immediate 8% mortality, which increases to 23% within the first year after the exacerbation (Groenewegen K H, et al., Chest 124:459-467, 2003). Recovery from COPD exacerbations is prolonged over weeks. Improvement in lung mechanics may require up to 6 weeks (Steven N J, et al., Am J Respir Crit Care Med 172:1510-1516, 2005). In a recent study of subjects suffering a COPD exacerbation, symptoms of shortness of breath, cough and sputum production failed to recover to pre-illness baseline in over 23% of subjects, even when measured up to 35 days after the onset of the exacerbation (Perera W R, et al., Eur Respir J 29:527-534, 2007). The greatest improvement in symptoms occurs in the first 4 weeks following onset of exacerbations, but some individuals are not fully recovered even by 26 weeks (Spencer S, et al., Thorax 58:589-593, 2003).

According to the National Heart, Lung, and Blood Institute, COPD is the fourth leading cause of death in the U.S., affects 10.7 million adults and annually costs $38.8 billion in 2005 dollars (Foster TS, et al., COPD 3:211-218, 2006). The largest portion of total expenditures (over 70%) is for inpatient hospitalization for exacerbations (Sullivan S D, et al., Chest 117:5-9, 2000; Ramsey S D, et al., Eur Respir J 21:29 S-35S, 2003). This is explained by the high cost of hospitalization for medical care. In the recent medical literature, the average length of hospital stay for patients with COPD exacerbations ranges from 5.9 days to 12 days (see Table 1). Any therapy effective at improving patients' airways dysfunction would allow physicians to discharge patients from the hospital sooner, greatly reducing the overall economic burden of COPD.

TABLE 1

| | | Average |
|---|---|---|
| | Admissions | Length |
| | for COPD | of Stay |
| Literature Document | Exacerbation | (days) |
| Saynajakangas O, et al., Age | 76,672 | 6.8 |
| and Ageing 33: 567-570, 2004 | | |
| Sala E, et al., Eur Respir J | 205 | 5.9 |
| 17: 1138-1142, 2002 | | |
| Sullivan S D, et al., Chest | 203,193 | 9.9 |
| 117: 5-9, 2000 | | |
| Kinnunen T, et al., Resp Med | 152,569 | 7.7 (without |
| 97: 143-146, 2003 | | comorbidity) |
| | | 10.5 (with |
| | | secondary |
| | | diagnosis) |
| Keistinen T, et al., Public | 188,570 | 9.6 (median |
| Health 110: 257-259, 1996 | | 7) |
| Kinnunen T, et al., Resp Med | 35,814 | 8.4 |
| 101: 294-299, 2007 | | |
| Connolly M J, et al., Thorax | 7,514 (247 | 8.7 |
| 61: 843-848, 2006 | hospitals) | |
| Price L C, et al., Thorax 61: 837- | 7,529 | 8.7 |
| 842, 2006 | | |
| Yohannes A M, et al., Age and | 100 | 12 (for |
| Ageing 34: 491-496, 2005 | | surviving |
| | | patients) |
| | | 21 (for |
| | | patients |
| | | who died) |
| McGhan R, et al. Chest | 51,353 | 6.5 |
| 132: 1748-1755, 2007 | | |

Because no curative therapy is available, management of severe exacerbations of COPD are generally directed at relieving symptoms and restoring functional capacity. Pharmacological management, such as recommended by ATS, includes the use of bronchodilators, anticholinergics, corticosteroids, antibiotics, and methylxanthines, as well as oxygen therapy and non-invasive ventilation (McCrory D C, et al., Chest 119:1190-1209, 2001; and Bach PB, et al., Ann Intern Med 132:600-620, 2001).

Bronchodilators are used to treat the increased breathlessness that occurs during exacerbations of COPD. Inhaled beta₂ agonists are typically administered (such as with nebulizers, hand-held metered dose, or dry powder inhalers) as soon as possible during an acute exacerbation. Specific examples of beta₂ agonists include albuterol, salbutamol, fomoterol, and terbutaline. Inhaled anticholingergics (such as ipratropium and tiotroprium) may also be used for bronchodilation and can also be administered by a nebulizer, metered-dose inhalers, or dry powdered inhaler. Combination products, such as ipratropium-albuterol (COMBIVENT®), are used to simplify the medication regimen.

In the absence of significant contraindications, oral corticosteroids are typically recommended, often in conjunction with other therapies, in all patients suffering from acute exacerbation of COPD. For severe exacerbations requiring inpatient therapy, prednisolone or methylprednisolone is commonly used.

Although bacteria can often be isolated from sputum samples during periods of COPD stability in patients, the presence of bacteria is also associated with exacerbations of COPD. Thus, antibiotics are often prescribed for exacerbations, particularly episodes of purulent sputum. Nevertheless, there has been controversy about whether antibiotics have a benefit in exacerbations, particularly episodes without purulent sputum. Moreover, as multiple agents have been associated with exacerbations of COPD, the type of antibiotic administered must be tailored to the specific infection underlying the exacerbation. Antibiotic resistance also poses an increasing problem, especially in infections caused by betalactamase-producing Haemophilus influenzae, Streptococcus pneumoniae, and Moraxella catarrhalis. Consequently, physicians often are forced to use broader spectrum antibiotics for empiric therapy. Some of the most commonly used antibiotics include: doxycycline, trimethoprim-sulfamethoxazole, amoxicillin-clavulanate potassium, clarithromycin, azithromycin, levofloxacin, gatifloxacin, moxifloxacin, ceftriaxone, cefotaxime, ceftazidime, piperacillin-tazobactam, ticarcillin-clavulanate potassium, and tobramycin.

Methylxanthines have an apparent action as bronchodilators and also exhibit action for increasing respiratory drive, thus making them apparently useful for overcoming some of the respiratory depression present during acute exacerbations of COPD. The use of methylxanthines, such as theophylline and aminophylline, is somewhat controversial. Although they can be of some help in improving diaphragmatic function, methylxanthines are potentially toxic and are associated with serious drug effects, including cardiac rhythm disturbances.

Despite the numerous pharmacologic treatments indicated for acute exacerbations of COPD, none of the known treatments have shown great success. As noted above, antibiotics can be successful in short-term outcomes, but there is no one “best” antibiotic, and long-term effects are questionable, particularly in the prevalence of antibiotic resistance. The other known treatments are generally directed toward either inhibiting the airway inflammation leading to the release of products that inhibit M₂ muscarinic autoreceptors on the postganglionic nerves or the M₃ muscarinic receptors on airway smooth muscle.

Acute exacerbations of COPD, particularly when arising from exposure to allergens, pollutants, or inhaled irritants, can be related to increased airway irritation and inflammation from the release of acetylcholine by cholinergic efferent motor branches of the vagus nerve (FIG. 1). In the airway, release of acetylcholine from the vagus nerves is under the local control of the M₂ and M₃ muscarinic receptors. Thus, acetylcholine released from the vagus nerve stimulates both M₃ muscarinic receptors on airway smooth muscle, causing bronchoconstriction, and M₂ muscarinic receptors on the nerves, decreasing further release of acetylcholine.

Corticosteroids are the mainstay of anti-inflammatory therapy, but the use thereof in the treatment of acute exacerbations of COPD is complicated by side effects (such as muscle weakness and increased catabolic state), and the best course of corticosteroid treatment is uncertain. Beta-adrenergic agonists, acting by stimulation of beta₂ adrenergic receptors on airway smooth muscle, are used as bronchodilators to directly reverse constricted airways. Nonselective anti-cholinergic drugs, such as ipratropium bromide, are available for use as bronchodilators, but block both prejunctional M₂ receptors and M₃ receptors on smooth muscle with equal efficacy. This increases acetylcholine release, overcoming the postjunctional blockade, and makes these nonselective anti-cholinergic agents ineffective at reversing vagally mediated bronchoconstriction. A more specific treatment for reversing the M₂ receptor blockade would be of great benefit as a treatment for the airway irritation and inflammation common with acute exacerbations of COPD.

The anticoagulant drug heparin has been shown to reverse antigen-induced M₂ receptor dysfunction in antigen-challenged guinea pigs (A. D. Fryer, et al., Journal of Clinical Investigation (1992) 90:2292-2298) and to reverse binding of M₂ receptor by major basic protein it vitro (D. B. Jacoby, et al., Journal of Clinical Investigation (1993) 91:1314-1318). However, heparin is an anticoagulant, and the use thereof in the treatment of acute exacerbations of COPD would expose the treated patient to an unacceptable risk of hemorrhage, even if treatment was localized by aerosolization of heparin into the lung airway. Aerosolized heparin is well absorbed into the systemic circulation, and administration of heparin by lung aerosolization has been advocated as a method of anticoagulating the blood (L. B. Jaques, et al., Lancet (1976) ii: 157-1161).

To use heparin safely as a treatment for acute exacerbations of COPD, it would need to be first inactivated as an anticoagulant without affecting its efficacy to treat acute exacerbations of COPD. Most know chemical methods for inactivating heparin as an anticoagulant are based on techniques of chemical desulfation, since it is well established that sulfate groups of heparin are important for anticoagulant activity. However, N-desulfated heparin has been previously reported to be only 50% as effective as heparin in complement inhibition (J. M. Weiler et al., J. Immunol. (1992) 148:3210-3215; R. E. Edens et al. Complement Today (Cruse, J. M. and Lewis, R. E. Jr. eds): Complement Profiles (1993)1:96-120). The present invention discloses that, however, selective O-desulfation of heparin eliminates the anticoagulant activity of heparin without destroying the ability of heparin to reverse the M₂ muscarinic receptor blockade.

Activated neutrophils play an important role in a number of human and other mammalian diseases by releasing a number of oxidant chemicals and enzymes after migration into an affected organ. While at least 21 separate destructive enzymes can be released, the major destructive elements produced by activated neutrophils are cationic proteases, the bulk of which consist of elastase and cathepsin G. When neutrophils release these proteases, tissue destruction occurs unless the proteases are neutralized by sufficient extracellular anti-proteinases, such as α-1-anti-proteinase.

As previously pointed out, cigarette smoking is implicated in 90% of diagnosed cases of COPD. Cigarette smoking causes an influx of activated leukocytes into the lungs with subsequent degranulation and release of proteases. Cigarette-derived oxidants inactivate α-1-anti-proteinase by oxidizing an important methionine near the active site. Elastase delivered to the alveolar lung unit as a result of the influx due to cigarette smoking, concurrent with oxidative inactivation of α-1-anti-proteinase activity, produces an imbalance of protease/anti-proteinase activity that is thought to be a major cause of human emphysema from cigarette smoking. Similarly, individuals with an inherited deficiency of α-1-anti-proteinase suffer unimpeded proteolytic lung destruction over a lifetime, resulting ultimately in the development of pulmonary emphysema.

When the imbalance of protease/anti-proteinase activity occurs within the airway, chronic airway inflammation is the result, and neutrophil derived elastase and cathepsin G are thought important in the pathogenesis of chronic bronchitis. If the imbalance occurs within the pulmonary vasculature, the resulting microvascular injury causes lung edema formation. In this fashion the influx of activated leukocytes and release of elastase and other neutrophil proteases are major causes of lung injury in the Adult Respiratory Distress Syndrome. Neutrophil derived elastase is also an important cause of proteolytic lung destruction in cystic fibrosis, a disease characterized by intense mucopurulent bronchitis and some of the highest levels of elastase activity measured in any human disease.

Because elastase and cathepsin G are mediators of a variety of important human diseases, developing effective inhibitors of these enzymes is an active goal in experimental pharmacology. Previous research has indicated O-desulfated heparin has elastase and cathepsin G inhibition activity. See, for example, U.S. Pat. No. 6,077,683, U.S. Pat. No. 5,912,237, U.S. Pat. No. 5,707,974, and U.S. Pat. No. 5,668,118, all of which are incorporated herein by reference in their entirety. This activity was unexpected since prior desulfation attempts that resulted in a decreased anticoagulant activity also resulted in a lack of elastase and cathepsin G inhibition activity.

The most useful approach to inhibiting elastase activity in the lung with an O-desulfated heparin is direct aerosolization into the lung so that O-desulfated heparin might directly combine with elastase released into the lung environment by activated neutrophils which migrate into the lung parenchyma and airways. While inhibition of elastase would be beneficial in COPD exacerbations, it would not prevent airway injury from release of destructive neutrophil oxidants such as hypochlorous acid (HOCl), since neither anticoagulant nor non-anticoagulant heparins are known to scavenge HOCl and prevent its injurious oxidant effects on tissue. The most effective method for reducing neutrophil injury in the lung would be to retard neutrophil migration into the lung from the blood stream, before neutrophils become activated and release proteolytic enzymes and oxidants into the lung environment. To accomplish this goal, one would employ an intravenous drug that retards neutrophil migration into the lung, thereby decreasing overall lung and systemic inflammation from COPD exacerbations. Currently, corticosteroids are used for this purpose, but have disadvantages, as previously discussed, including the induction of muscle weakness, an increased catabolic state, the induction of osteoporosis, induction of elevated blood pressure, and the induction of glucose resistance leading in some cases to the diabetic state. The anti-inflammatory effect of corticosteroids in COPD exacerbations is also modest, and leads to only moderate reductions in systemic inflammation measured by CRP and decreases in length of hospital stay to no shorter than an average 8.5 days in the only large, randomized controlled trial of these agents. No other drug which decreases neutrophil migration into the lung, decreases lung or systemic inflammation during COPD exacerbations, or decreases length of hospital stay is known to exist. To have such an agent free of the side effects of corticosteroids would be a major pharmacologic advance in treating COPD exacerbations.

One major problem in using heparin or heparin-derived agents to treat inflammation from COPD exacerbations is that heparin and its derivatives cause heparin-induced thrombocytopenia (HIT), a disastrous fall in platelet count produced by the formation of a complex between heparin and platelet factor 4 (PF-4), a 70-amino acid platelet specific chemokine found in platelet granules. When heparin binds to PF-4, it produces a conformational change in PF-4, exposing an antigenic epitope to which some few individual have a circulating antibody (HIT antibody). The HIT antibody binds heparin-PF-4 complexes with high affinity. This antibody-heparin-PF-4 complex then binds to platelets by attachment of the antibody Fc domain to the platelet Fc receptor (FcγRIIa). This event in turn cross-links the Fc platelet receptors, inducing platelet activation and aggregation. A wave of platelet activation then ensues, producing consumption of platelets, a fall in platelet count to less than 50% of baseline (thrombocytopenia) and generalized coagulation, with potential development of life-threatening venous and arterial thrombosis, which can produce pulmonary embolism, myocardial infarction, stroke, or loss of limb perfusion. Any person receiving heparin or a heparin-like molecule is normally at risk for developing the type II heparin-induced thrombocytopenia that is associated with the risk of subsequent platelet-induced thrombosis. The overall risk for developing type II HIT is 0.5 to 3.0% of patients given heparin or a heparinoid (Chong, B H, et al., Expert Review of Cardiovascular Therapy 2:547-559, 2004).

### SUMMARY OF THE INVENTION

In light of the activity of O-desulfated heparin to reverse the M₂ muscarinic receptor blockade, as well as inhibit elastase and cathepsin G, it has surprisingly been found according to the present invention that O-desulfated heparin is particularly useful in methods of treating and preventing acute exacerbations of COPD. As previously pointed out, COPD is generally recognized as a disease state characterized by airflow limitation that is not fully reversible, and is in fact a progressive disease. As evidenced by the present invention, acute exacerbation of COPD is not merely a progression of the irreversible COPD but is rather an episodic worsening of the condition that can be reversed to the baseline condition of the patient with COPD.

Accordingly, it has been discovered according to the present invention that O-desulfated heparin can be used in the treatment and prevention of acute exacerbations of COPD. Specifically, without dangerous anticoagulation of the blood, which would place patients at risk from complications of bleeding, O-desulfated heparin is particularly effective at decreasing the degree of pulmonary and systemic inflammation during COPD exacerbations, as measured by CRP, and O-desulfated heparin improves resolution of the COPD exacerbation, allowing patients to be discharged from the hospital earlier than noted in the medical literature of COPD exacerbations. In certain embodiments, patients suffering from COPD exacerbation, when treated with O-desulfated heparin according to the invention, can be released from the hospital a full day earlier than the shortest length of hospital stay reported in the medical literature around COPD exacerbations. It is a major advantage that O-desulfated heparins can reduce lung and systemic inflammation in COPD exacerbations without producing the unwanted side effects of corticosteroids, including elevated glucose, a catabolic state, muscle weakness, and elevated blood pressure. It is another major advantage that O-desulfated heparin, specifically heparin desulfated at the 2-O position, does not produce HIT, and can decrease lung and systemic inflammation during COPD exacerbations without producing HIT or profound thrombocytopenia.

That O-desulfated heparin can be used in the treatment of acute exacerbations of COPD is particularly surprising because the widespread acceptance of the irreversibility of COPD generally. Asthma is distinctly separated from COPD by the reversibility of the asthmatic episodes. As disclosed in U.S. Pat. No. 5,990,097 (which is incorporated herein by reference), O-desulfated heparin is useful in reversing the underlying causes of the airway hyperactivity associated with the asthmatic episodes. Prior to the present invention, it was believed that acute exacerbations of COPD could only be managed, not treated. Accordingly, routine interventions for acute exacerbations, as previously described, generally include bronchodilators, antibiotics, and steroids. Thus, management is directed at fighting bacterial agents and reducing inflammation in the airways. The present invention, however, realizes the ability to actually treat and prevent acute exacerbations by using O-desulfated heparin to act on the physiological pathways causing the inflammation and worsening the condition.

Accordingly, the present invention provides a method of treating a patient suffering from an acute exacerbation of COPD. In one embodiment, the method of the invention comprises administering to the patient a pharmaceutical composition comprising O-desulfated heparin. In preferred embodiments, the composition comprises O-desulfated heparin in a treatment effective amount, which is an amount useful to lessen or eliminate the acute exacerbation of COPD. In yet further preferred embodiments, the O-desulfated heparin is O-desulfated at least at the 2-O and 3-O positions.

The presence of an acute exacerbation can be determined based upon the presence of one or more symptoms typically recognized as being indicative of an acute exacerbation of COPD. In certain embodiments, an acute exacerbation is indicated by the presence of a symptom selected from the group consisting of increased sputum production, more purulent sputum, change in sputum color, increased coughing, increased wheezing, chest tightness, reduced exercise tolerance, increased fatigue, fluid retention, acute confusion, worsened dyspnea, and combinations thereof. Thus, the method of the invention for lessening or eliminating an acute exacerbation can comprise lessening or eliminating a symptom of an acute exacerbation of COPD. Moreover, a treatment effective amount of O-desulfated heparin can be an amount effective to lessen or eliminate a symptom of an acute exacerbation of COPD.

The invention is characterized in that the O-desulfated heparin can be administered to a patient after onset of an acute exacerbation of COPD in the method for treating the exacerbation. Alternatively, the O-desulfated heparin can be administered to a patient having COPD prior to an exacerbation to prevent onset of an exacerbation.

The inventive methods of treatment can further include, in addition to the O-desulfated heparin, one or more additional active agents. Such additional agents can be any agent recognized as useful in the treatment or management of COPD or acute exacerbations of COPD. In certain embodiments, the one or more additional active agents are selected from the group consisting of bronchodilators, anticholinergics, corticosteroids, antibiotics, methylxanthines, and combinations thereof. Such additional active agents can be combined with O-desulfated heparin in a single composition or can be co-administered with O-desulfated heparin as separate compositions.

Administration of the composition to effect treatment according to the invention can be by a variety of routes. For example, in certain embodiments, administration is via a route selected from the group consisting of intravenous administration, subcutaneous administration, inhalation, and combinations thereof. In specific embodiments, the methods of the invention comprise administering the composition as a bolus. Administration can also comprise constantly infusing the composition for a predetermined time, such as about 12 hours to about 168 hours. In specific embodiments, the inventive methods comprise administering a bolus of the composition followed by constantly infusing the composition for a predetermined time.

Treatment of the acute exacerbation by administration of O-desulfated heparin, as described herein, can be evidenced by various outcomes. For example, as noted above, treatment can comprise lessening or eliminating a symptom of an acute exacerbation of COPD.

Further, it is common for a patient suffering an acute exacerbation of COPD to require hospitalization. In such cases, treatment according to the invention can be effected such that hospitalization time of the patient is less than typically required when no treatment with O-desulfated heparin is provided. In specific embodiments, hospitalization is less than five days, preferably less than four days. In other embodiments, treatment can be effected such that the patient achieves a reduction in lung inflammation, which can be evidenced by a reduction in measured levels of plasma C-reactive protein (CRP). In specific embodiments, treatment is effected such that CRP is reduced by at least about 60% in a time of less than 120 hours after administration of the composition according to the invention. Thus, the ability to lessen or eliminate an acute exacerbation of COPD according to the methods of the invention can comprise such a reduction in hospitalization.

Accordingly, in specific embodiments, the invention provides a method of reducing hospitalization time for a patient suffering from an acute exacerbation of COPD. The method preferably comprises administering to the patient a pharmaceutical composition comprising an amount of O-desulfated heparin effective to treat the acute exacerbation. According to this embodiment, the hospitalization time for the patient is less than the hospitalization time for a patient suffering from an acute exacerbation of COPD but not treated with the O-desulfated heparin. In specific embodiments, hospitalization time is reduced by at least about 10% in comparison to a patient suffering an acute exacerbation of COPD requiring hospitalization but not treated with O-desulfated heparin.

An acute exacerbation of COPD is typically evidenced by lung inflammation. Thus, the treatment of acute exacerbations of COPD according to the invention can also comprise reducing such lung inflammation. In certain embodiments, the present invention thus provides methods for reducing lung inflammation in a patient suffering from an acute exacerbation of COPD. Preferentially, the method comprises administering to the patient a pharmaceutical composition comprising an amount of O-desulfated heparin effective to reduce lung inflammation. The reduced inflammation can particularly be evidenced as a decrease in the plasma C-reactive protein (CRP) of the patient. In specific embodiments, measured plasma CRP is reduced by at least about 60%. The desired reduction is preferably achieved within less than 168 hours from the onset of treatment according to the invention.

### BRIEF DESCRIPTION OF THE DRAWINGS

FIG. 1 is a schematic drawing of cholinergic neural pathways and muscarinic receptor subtypes of the afferent sensory and efferent motor limbs of the vagus nerve innervation of the lung airway, wherein Ach is acetylcholine, N is nicotinic receptor, M₁ is M₁ muscarinic receptor, M₂ is M₂ muscarinic receptor, M₃ is M₃ muscarinic receptor, and arrows indicate neurotransmission;

FIG. 2 is a chemical formula of the pentasaccharide binding sequence of unfractionated heparin (top formula) and the comparable sequence of 2-O, 3-O desulfated heparin (ODS heparin or ODSH) (bottom formula);

FIG. 3 is a graph showing inhibition by 2-O, 3-O desulfated heparin of human monocyte attachment to P-selectin;

FIG. 4 is a graph of mean plasma concentrations of O-desulfated heparin in normal human subjects receiving a bolus dose of the agent intravenously;

FIG. 5 is a graph of mean change from baseline in activated partial thromboplastin time (aPTT) in normal human subjects receiving an intravenous bolus dose of O-desulfated heparin;

FIG. 6 is a graph of mean plasma concentrations of O-desulfated heparin in normal human subjects receiving a bolus followed by 12 hour infusion of the drug;

FIG. 7 is a graph of mean change from baseline in activated partial thromboplastin time (aPTT) in normal human subjects receiving an intravenous bolus dose and 12 hour infusion of O-desulfated heparin;

FIG. 8 is a graph of mean plasma levels of O-desulfated heparin (ODSH) in subjects receiving an intravenous bolus of 8 mg/kg O-desulfated heparin followed by an infusion of 0.6 mg/kg/hr for 72 hours, titrated to maintain aPTT at the upper limit of normal (ULN) in the range of 40-45 seconds;

FIG. 9 is a graph of mean activated partial thrombopastin time (aPTT) in normal human subjects receiving an intravenous bolus of 8 mg/kg O-desulfated heparin followed by an infusion of 0.6 mg/kg/hr for 72 hours, titrated to maintain aPTT at the upper limit of normal (ULN) in the range of 40-45 seconds;

FIG. 10 is a graph showing the relationship between plasma levels of O-desulfated heparin (ODSH) and change in activated partial thromboplastin time (aPTT) from baseline in normal human subjects receiving an intravenous bolus of 8 mg/kg O-desulfated heparin followed by an infusion of 0.6 mg/kg/hr for 72 hours, titrated to maintain aPTT in the upper limit of normal (ULN) in the range of 40-45 seconds;

FIG. 11 is a graph of activated partial thromboplastin times (aPTT) in human subjects hospitalized with COPD exacerbations who received an intravenous bolus of O-desulfated heparin (ODSH) of 8 mg/kg followed by an infusion of 0.5 mg/kg/hr for 72 hours, or until the patient's COPD exacerbation had improved sufficiently to allow hospital discharge; and

FIG. 12 is a graph of plasma C-reactive protein (CRP) concentrations in human subjects hospitalized with COPD exacerbations and administered O-desulfated heparin (ODSH) in an intravenous bolus dose of 8 mg/kg followed by an infusion of 0.5 mg/kg/hr for 72 hours, or until the patient's COPD exacerbation had improved sufficiently to allow hospital discharge.

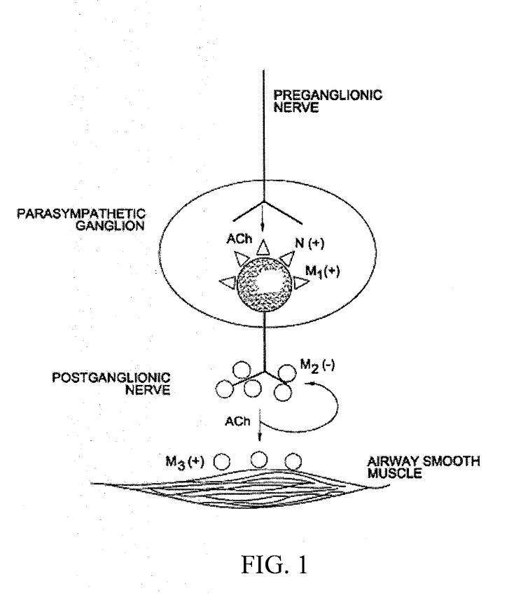

### DETAILED DESCRIPTION OF THE INVENTION

The present inventions now will be described more fully hereinafter with reference to specific embodiments of the invention and particularly to the various drawings provided herewith. Indeed, the invention may be embodied in many different forms and should not be construed as limited to the embodiments set forth herein; rather, these embodiments are provided so that this disclosure will satisfy applicable legal requirements. As used in the specification, and in the appended claims, the singular forms “a”, “an”, “the”, include plural referents unless the context clearly dictates otherwise.

### I. Active Agents

The present invention provides pharmaceutical compositions useful in methods of treatment of acute exacerbations of COPD. The pharmaceutical compositions of the invention generally comprise O-desulfated heparin (ODSH) as an active agent. In certain embodiments, the pharmaceutical compositions can comprise one or more further active agents.

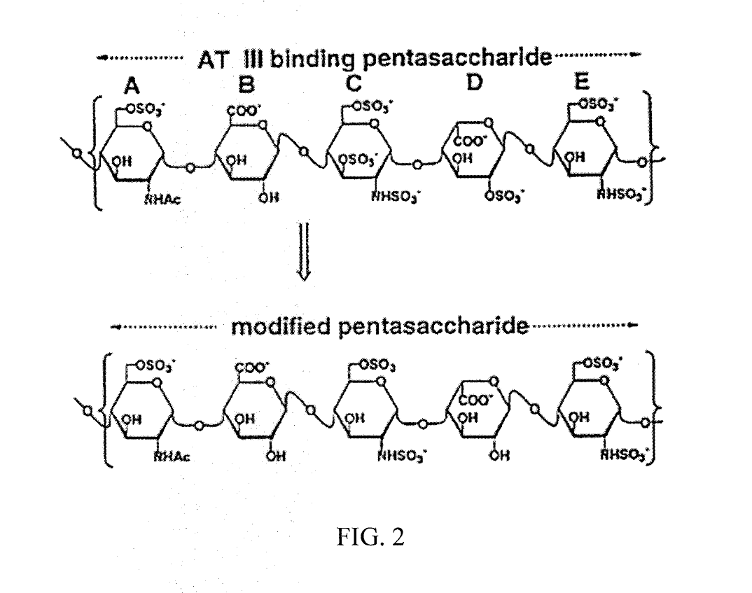

The chemical formula of naturally occurring heparin is shown in the top formula provided in FIG. 2. Modified heparin, or O-desulfated heparin, is illustrated in the bottom formula in FIG. 2. The term “O-desulfated heparin” refers to heparin that has been modified to remove at least a portion of the O-sulfate groups therefrom. Preferably, the term refers to heparin that is O-desulfated sufficiently to have resulted in any reduction of the anticoagulant activity of the heparin. In specific embodiments, the O-desulfated heparin is at least partially, and preferably substantially, desulfated at least at the 2-O position, at least at the 3-O position, or at both the 2-O position and the 3-O position.

In preferred embodiments, the O-desulfated heparin is at least about 10%, at least about 25%, at least about 50%, at least about 75%, at least about 80%, at least about 90%, at least about 95%, at least about 97%, or at least about 98% desulfated, independently, at each of the 2-O position and the 3-O position. In specific embodiments, the O-desulfated heparin is 100% desulfated at one or both of the 2-O and the 3-O position. The extent of O-desulfation need not be the same at each O-position. For example, the heparin could be predominately (or completely) desulfated at the 2-O position and have a lesser degree of desulfation at the 3-O position (or vice-versa). In one embodiment, the O-desulfated heparin comprises 2-O, 3-O desulfated heparin, wherein the heparin is at least about 90% desulfated at both the 2-O and 3-O positions.

The extent of O-desulfation or N-desulfation can be determined by known methods, such as disaccharide analysis. Although 6-O desulfation cannot be determined by currently available techniques, in a preferred embodiment, the 6-O position is substantially sulfated. Of course, the invention still encompasses heparin wherein some, particularly a minor amount, of the 6-O sulfates were lost (desulfated) during the preparation of the compounds used in the invention. N-sulfates are generally stable under alkaline hydrolytic conditions. Thus, in certain embodiments, the heparin used according to the invention can have most of its N-sulfate groups remaining intact. Of course, the invention does encompass heparin having some of the N-sulfates removed.

One method of preparing O-desulfated heparin is provided in U.S. Pat. No. 5,990,097. In the method disclosed therein, a 5% aqueous solution of porcine intestinal mucosal sodium heparin is made by adding 500 gm heparin to 10 L deionized water. Sodium borohydride is added to a 1% final concentration and the mixture is incubated. Sodium hydroxide is then added to a 0.4 M final concentration (pH at least 13) and the mixture is frozen and lyophilized to dryness. Excess sodium borohydride and sodium hydroxide can be removed by ultrafiltration. The final product is pH adjusted, cold ethanol precipitated, and dried. The O-desulfated heparin produced by this procedure is a fine crystalline slightly off-white powder with less than 10 USP units/mg anti-coagulant activity and less than 10 U/mg anti-Xa anti-coagulant activity.

The synthesis of O-desulfated heparin as described above can also include various modifications. For example, the starting heparin can be place in, for example, water, or other solvent, as long as the solution is not highly alkaline. A typical concentration of heparin solution can be from 1 to 10 percent by weight heparin. The heparin used in the reaction can be obtained from numerous sources, known in the art, such as porcine intestine or beef lung. The heparin can also be modified heparin, such as the analogs and derivatives described herein.

The heparin can be reduced by incubating it with a reducing agent, such as sodium borohydride, catalytic hydrogen, or lithium aluminum hydride. A preferred reduction of heparin is performed by incubating the heparin with sodium borohydride. Generally, about 10 grams of NaBH₄ can be used per liter of solution, but this amount can be varied as long as reduction of the heparin occurs. Additionally, other known reducing agents can be utilized but are not necessary for producing a treatment effective O-desulfated heparin. The incubation can be achieved over a wide range of temperatures, taking care that the temperature is not so high that the heparin caramelizes. Exemplary temperature ranges are about 15-30° C. or about 20-25° C. The length of the incubation can also vary over a wide range, as long as it is sufficient for reduction to occur. For example, several hours to overnight (i.e., about 4 to 12 hours) can be sufficient. However, the time can be extended to over several days, for example, exceeding about 60 hours.

Additionally, the method of synthesis can be adapted by raising the pH of the reduced solution to 13 or greater by adding a base capable of raising the pH to 13 or greater to the reduced heparin solution. The pH can be raised by adding any of a number of agents including hydroxides, such as sodium, potassium or barium hydroxide. A preferred agent is sodium hydroxide (NaOH). Even once a pH of 13 or greater has been achieved, it can be beneficial to further increase the concentration of the base. For example, it is preferable to add NaOH to a concentration of about 0.25 M to about 0.5 M NaOH. This alkaline solution is then dried, lyophilized or vacuum distilled.

In specific embodiments, the alkaline solution can comprise heparin and base in defined ratios. For example, when NaOH is used as the base, the ratio of NaOH to heparin (NaOH:heparin, in grams) can be about 0.5:1, preferably about 0.6:0.95, more preferably about 0.7:0.9. Of course, greater concentrations of base can be added, as necessary, to ensure the pH of the solution is at least 13.

Heparin is a heterogeneous mixture of variably sulfated polysaccharide chains composed of repeating units of D-glucosamine and either L-iduronic acid or D-glucuronic acids. The average molecular weight of heparin typically ranges from about 6,000 Da to about 30,000 Da, although certain fractions of unaltered heparin can have a molecular weight as low as about 1,000 Da. According to certain embodiments of the invention, heparin can have a molecular weight in the range of about 1,000 Da to about 30,000 Da, about 3,000 Da to about 25,000 Da, about 8,000 Da to about 20,000 Da, or about 10,000 Da to about 18,000 Da. Unless otherwise noted, molecular weight is expressed herein as weight average molecular weight (M_{w}), which is defined by formula (I) below

$${M}_{W} = \frac{\sum {n}_{i} {M}_{i}^{2}}{\sum {n}_{i} {M}_{i}} , \qquad ( I )$$

wherein nᵢ is the number of polymer molecules (or the number of moles of those molecules) having molecular weight Mᵢ.

The O-desulfated heparin used according to the invention can also have a reduced molecular weight so long as it retains the useful activity as described herein. Low molecular weight heparins can be made enzymatically by utilizing heparinase enzymes to cleave heparin into smaller fragments, or by depolymerization using nitrous acid. Such reduced molecular weight O-desulfated heparin can typically have a molecular weight in the range of about 100 Da to about 8,000 Da. In specific embodiments, the heparin used in the invention has a molecular weight in the range of about 100 Da to about 30,000 Da, about 100 Da to about 20,000 Da, about 100 Da to about 10,000 Da, about 100 Da to about 8,000 Da, about 1,000 Da to about 8,000 Da, about 2,000 Da to about 8,000 Da, or about 2,500 Da to about 8,000 Da. Preferably, the average molecular weight of the heparin after O-desulfation is in the range of about 8,000 Da to about 12,500 Da.

As noted above, in certain embodiments, the methods of the invention can comprise the use of one or more active agents in addition to O-desulfated heparin. The additional active agent can be combined with O-desulfated heparin into a single composition. Alternately, the additional active agent can be provided as a separate composition that is co-administered with the O-desulfated heparin (e.g., administered at the same time or sequentially within a treat effective time frame, which could be only a few seconds or up to several hours).

Non-limiting examples of active agents that can be used with O-desulfated heparin for treatment of acute exacerbations of COPD include any drugs presently used in management of COPD generally or for treatment of acute exacerbations of COPD. For example, the additional active agent could be selected from bronchodilators (particularly beta-agonists), anticholinergics, corticosteroids, antibiotics, or methylxanthines. Of course, such disclosure should not be viewed as limiting the scope of further active agents that may be combined with O-desulfated heparin. Rather, any further compounds generally recognized as useful for treating acute exacerbations of COPD may be used in addition to the compounds specifically noted herein.

### II. Methods of Treatment

The present invention generally provides a method for the treatment of an acute exacerbation of COPD. It is well understood in the art that COPD is a long-term illness where a patient has an established baseline of reduced pulmonary function. It is likewise understood in the art that acute exacerbation of COPD is a distinct illness that is actually treated separately from the underlying COPD. The U.S. Agency for Healthcare Research and Quality (AHRQ), a division of the U.S. Department of Health and Human Services, provides clinical guidelines for the management of acute exacerbations of COPD (available online at http://www.ahrq.gov/clinic/epcsums/copdsum.htm). The AHRQ report specifically states that management of acute exacerbations of COPD excludes from consideration other conditions, such as asthma, cystic fibrosis, bronchiectasis, and stable COPD. Thus, the methods of the present invention in relation to treatment of acute exacerbations of COPD are distinct from methods of treating stable COPD.

An acute exacerbation of COPD is typically defined as a sustained worsening of the patient's symptoms from his or her usual stable state that is beyond normal day-to-day variations, and is acute in onset. In other words, an acute exacerbation of COPD is a noticeable change from the baseline condition of the patient with COPD. Thus, the method of the invention can be described as treatment of a patient with COPD wherein the patient is experiencing an acute exacerbation of the condition exhibiting one or more symptoms that are acutely worsened from the baseline condition of the patient.

Acute exacerbations of COPD typically manifest as increased sputum production, more purulent sputum, change in sputum color, increased coughing, upper airway symptoms (e.g., colds and sore throats), increased wheezing, chest tightness, reduced exercise tolerance, increased fatigue, fluid retention, acute confusion, and worsening of dyspnea. Thus, in certain embodiments, the invention provides a method of treating an acute exacerbation of COPD in a patient, wherein the exacerbation is manifested by one or more of the symptoms noted above. In still further embodiments, the invention provides methods of treating one or more symptoms of an acute exacerbation of COPD.

In preferred embodiments, the inventive method is useful to lessen or eliminate a symptom of acute exacerbation, such as the symptoms described above. In one embodiment, the invention is useful to lessen or eliminate dyspnea, particularly dyspnea that is worsened from a normally present dyspnea. In other embodiments, the method is useful to lessen or eliminate increased sputum purulence, particularly clearing the sputum from purulent. The method is further useful to lessen or eliminate an increased cough, lessen or eliminate bronchoconstriction, and lower elevated lung volumes present during an acute exacerbation of COPD. In other embodiments, the inventive method is useful to lessen or eliminate increased wheezing, lessen or eliminate chest tightness, lessen or eliminate increased fatigue, lessen or eliminate increased fluid retention, and lessen or eliminate acute confusion. In still further embodiments, the method of the invention is useful to improve reduced exercise tolerance during an exacerbation, particularly in comparison to the exercise tolerance of patients suffering from an acute exacerbation of COPD who are not being treated according to the methods of the invention.

In certain embodiments, the methods of treatment according to the invention generally comprise administering O-desulfated heparin to a patient suffering from an acute exacerbation of COPD. Such an exacerbation can be determined by the presence of one or more of the symptoms of an exacerbation described above, as well as any further symptoms generally recognized as signaling an acute exacerbation of COPD.

The methods of the invention, in addition to treating a patient suffering from an acute exacerbation of COPD, also provide for preventing an exacerbation in a patient suffering from COPD. Thus, the invention encompasses administering to a patient having COPD, but not actively exhibiting symptoms of an exacerbation thereof, an amount of O-desulfated heparin effective to prevent the onset of an acute exacerbation of COPD.

By “prevention” is meant that the patient suffering from COPD does not develop one or more of the symptoms described herein in relation to an acute exacerbation of the disease at an acute level. Preferably exacerbations, as manifested by the symptoms described herein, are completely avoided. For prevention, the O-desulfated heparin can be administered prior to exposure to an exacerbation stimulus, such as prior to a predicted contact with a known antigen or a place presenting such antigens. Also, the O-desulfated heparin can be administered on a routine basis to continually prevent exacerbations.

Preferably a prevention method of this invention comprises a constant suppression of the symptoms indicating an acute exacerbation of COPD, which can be achieved by a repetitive, routine administration of the O-desulfated heparin. With repetitive, routine administration, an optimal dose can readily be ascertained by varying the dose until the optimal prevention is achieved. Additionally, upon exposure to large amounts of an antigen or irritant, if eventually one or more symptoms of an exacerbation occur, an additional dose of O-desulfated heparin can be administered. Additionally, when an exposure to a large antigen amount is known in advance, an additional dose of O-desulfated heparin can be administered to prevent a response.

While not wishing to be bound by theory, it is believed that the use of heparin according to the invention is particularly useful since it blocks the influx of inflammatory leukocytes into the lung that may mediate the symptoms of COPD exacerbations. Additionally, heparin is useful for blocking the irritant sensory nerves in the submucosa of airways. When these nerves are triggered by inflammation, they start a reflex arc that ends in vagally mediated muscarinic bronchoconstriction. By blocking the sensory arc, heparin beneficially prevents bronchoconstriction, which is an underlying cause of dyspnea (i.e., bronchoconstriction causes enlarged lung volumes in COPD patients, which leads to shortness of breath).

In certain embodiments, the invention is directed to methods of reducing hospitalization time for a patient suffering from an acute exacerbation of COPD. As described above in relation to Table 1, recent medical literature indicates that the average length of hospital stay for patients with COPD exacerbations ranges from 5.9 days to 12 days (or an average of about 9 days). The present invention is particularly useful in that the methods of treatment described herein can significantly reduce such hospitalization. This is highly beneficial not only from the standpoint of reduced costs to the patient and the hospitals, but also for improving patient quality of life and avoiding excess exposure to the hospital environment where secondary infections can be readily acquired.

The Examples provided below illustrate the ability of the inventive methods for reducing hospitalization of patients suffering from acute exacerbations of COPD. This is particularly so for patients treated with conventional therapies in association with the treatments of the invention. In particular, the method for reducing hospitalization comprises administering to the patient a pharmaceutical composition comprising an amount of O-desulfated heparin effective to treat the acute exacerbation. Such treatment with O-desulfated heparin allows for hospitalization time that is less than the hospitalization time for a patient suffering from an acute exacerbation of COPD but not treated with the O-desulfated heparin (including patients treated with the conventional therapies of COPD). Average hospitalization time, as used herein, is measured as the time from the onset of treatment in the hospital to the time the exacerbation is sufficiently lessened or eliminated such that the patient is discharged from the hospital. This is typical a determination made by the attending physician based on the state of the patient's health.

In determining whether a patient is sufficiently recovered for hospital discharge, the methods of the invention can comprise the use of the Global Initiative for Chronic Obstructive Lung Disease (GOLD) recommended criteria for hospital discharge (Rabe K F, et al., Am J Respir Crit Care Med 176:532-555, 2007). The GOLD standard, to which most physicians typically adhere, generally requires that the patient is sufficiently recovered to meet the following standards:
1. Inhaled β₂-agonist therapy is required no more frequently than every 4 hours;
2. The patient, if previously ambulatory, is able to walk across the room;
3. The patient is able to eat and sleep without frequent awakening by dyspnea;
4. The patient has been clinically stable for 12-24 hours;
5. The patient's arterial blood gases have been stable for 12-24 hours
6. The patient (or the home caregiver) fully understands correct use of medications;
7. Follow-up and home care arrangements have been completed (e.g., visiting nurse, oxygen delivery, meal provisions); and
8. The patient, family, and physician are confident the patient can manage successfully at home.
   As seen above, the discharge determination includes both objective and subjective evaluations. The above standards, though, are sufficient so that a skilled person (e.g., a physician experienced in treating patients with COPD that suffer acute exacerbations) would be able to consistently evaluate the average length of time patients are treated until they are sufficiently recovered for discharge. Thus, a skilled person could easily evaluate whether a method of treatment by administering O-desulfated heparin is according to the present invention by determining the average length of hospitalization of patients so treated. In certain embodiments, the time of discharge of a patient is established when the patient meets at least one of the criteria noted above. In other embodiments, the time of discharge is established when the patient meets at least two, at least three, at least four, at least five, or at least six of the above criteria. In specific embodiments, the time of discharge of established when the patient meets all of the above criteria.

Whereas patients hospitalized for acute exacerbations of COPD typically require stays of 6-12 days, treatment according to the present invention allows for patient discharge in as little as 3 days, which is 3 days less than even the shortest hospitalization time provided in the literature. Moreover, patients treated according to the present invention have been discharged from hospitalization for exacerbation of COPD in an average of about 4 days. By contrast, the literature reports an average hospital stay commonly as long as 12 days. See, for example, Table 1.

In light of the above, it is clear that the methods of the invention, including treatment with O-desulfated heparin, hastens the time to improvement of the COPD exacerbation, including when added to the conventional standard of care therapy for such patients. In specific embodiments, treatment with O-desulfated heparin according to the present invention reduces the time of hospitalization of a patient suffering an acute exacerbation of COPD by at least about 10% compared to a patient suffering an exacerbation of COPD but not treated with O-desulfated heparin. In further embodiments, the time of hospitalization is reduced by at least about 15%, at least about 20%, at least about 25%, at least about 30%, at least about 40%, at least about 50%, or at least about 60%. In further embodiments, the reduced time of hospitalization can be described in terms of the number of days of hospitalization. In specific embodiments, treatment according to the invention reduces the average time of hospitalization of a patient suffering an exacerbation of COPD by at least 1 day. In further embodiments, the average time of hospitalization is reduced by at least 2 days, at least 3 days, or at least 4 days.

As noted above, the average time of hospitalization for a patient suffering from an acute exacerbation of COPD when treated according to conventional therapies alone is commonly as much as 12 days. The average time of hospitalization for patients suffering exacerbations of COPD when treated according to the present invention, as illustrated in Example 5, is about 4 days. Thus, the methods of the invention clearly show a reduction in the average time of hospitalization to less than 5 days, preferably less than 4 days. This is a decrease of as much as 8 days in comparison to the reported hospitalization time when conventional therapies alone are used.

In other embodiments, the invention is directed to methods for reducing lung inflammation in a patient suffering from an acute exacerbation of COPD. As described above, C-reactive protein (CRP) is the most commonly used biomarker of inflammation, including in the lungs. Thus, CRP can be used to monitor inflammation within the airways as it relates to a decline in lung function, and elevated CRP can be relied used in the following ways:

1) as a marker of the degree of airway inflammation in stable COPD;

2) as a marker of COPD exacerbation generally; and

3) as a marker of improvement in airways inflammation over time.

Accordingly, in specific embodiments, the methods of the invention comprise administering to a patient suffering from an acute exacerbation of COPD an amount of O-desulfated heparin effective to reduce lung inflammation. In particular, the reduced lung inflammation can be evaluated as a decrease in the measured plasma CRP of the treated patient.

As illustrated in the Examples, O-desulfated heparin reduces lung and systemic inflammation as measured by CRP. Normally, even with corticosteroid therapy (which is undesirable in light of its side-effects), CRP falls only by about 50% during the course of hospitalization for COPD exacerbations (which, as noted above, can be as long as 12 days). In previous studies, corticosteroid therapy over the course of seven days has been shown to reduce plasma CRP from an average of 10.9 mg/L to an average of 5.3 mg/L (a decrease of 51.4%).

By contrast, treatment according to the present invention has been shown to reduce plasma CRP in patients suffering from acute exacerbations of COPD by approximately 81%. For example, patients treated according to the invention were subject to CRP evaluations on the day of hospital admission, on day two of hospitalization, and on the day of hospital discharge (which ranged from three to six days from the day of hospitalization). In these patients treated according to the invention, plasma CRP levers were reduced from an average of 22.1 mg/L (+11.0 mg/L) on the day of hospital admission to an average of 4.2 mg/L (+3.4 mg/L) on the day of hospital discharge. This data is reported below in Table 16. In other words, treatment according to the present invention was shown useful for reducing average plasma CRP by greater than 80% within less than 144 hours. This indicates that treatment using O-desulfated heparin is particularly useful for reducing lung inflammation in patients suffering a COPD exacerbation.

Plasma CRP can be measured by any method generally recognized as useful for such measurement in the art. U.S. Pat. No. 6,406,862, which is incorporated herein by reference in its entirety, describes a dipstick method for measuring CRP in a fluid (such as plasma). Other CRP assays are know in the art and could be used for measuring and evaluating CRP levels according to the invention.

In specific embodiments of the invention, measured plasma CRP is reduced by at least about 50% following treatment according to the invention. In further embodiments, measured CRP is reduced by at least about 55%, at least about 60%, at least about 65%, at least about 70%, at least about 75%, at least about 80%, at least about 85%, or at least about 90% following treatment according to the invention. The noted reduction in CRP (and the accompanying reduction in lung inflammation) is preferably achieved within a given time from the first administration of the O-desulfated heparin to begin treatment of the acute exacerbation of COPD. In specific embodiments, the desired reduction in CRP is achieved in a time of less than 168 hours, less than 144 hours, less than 120 hours, less than 96 hours, less than 72 hours, or less than 60 hours. This time can be measured from the administration of a single dose (such as a bolus), can be measured from the time of first administration in an intermittent dosing regimen (such as periodic inhaler treatments), or can be measured from the onset of administration of a continuous treatment (such as a constant infusion).

According to one embodiment, the methods of the invention are useful for reducing average plasma CRP level by at least 50% in a time of less than about 168 hours, less than about 144 hours, less than about 120 hours, less than about 96 hours, less than about 72 hours, or less than about 60 hours. According to another embodiment, the methods of the invention are useful for reducing average plasma CRP level by at least 60% in a time of less than about 168 hours, less than about 144 hours, less than about 120 hours, less than about 96 hours, less than about 72 hours, or less than about 60 hours. According still another embodiment, the methods of the invention are useful for reducing average plasma CRP level by at least 70% in a time of less than about 168 hours, less than about 144 hours, less than about 120 hours, less than about 96 hours, less than about 72 hours, or less than about 60 hours.

### III. Biologically Active Variants

Biologically active variants of O-desulfated heparin are particularly also encompassed by the present invention and may be used in the methods disclosed herein. Such variants should retain the biological activity of the original compound; however, the presence of additional activities would not necessarily limit the use thereof in the present invention. Such activity may be evaluated using standard testing methods and bioassays recognizable by the skilled artisan in the field as generally being useful for identifying such activity.

According to one embodiment, suitable biologically active variants useful according to the invention comprise analogues and derivatives of the compounds described herein. Indeed, a single compound, such as those described herein, may give rise to an entire family of analogues or derivatives having similar activity and, therefore, usefulness according to the invention. Likewise, a single compound, such as those described herein, may represent a single family member of a greater class of compounds useful according to the present invention. Accordingly, the present invention fully encompasses not only the compounds described herein, but analogues and derivatives of such compounds, particularly those identifiable by methods commonly known in the art and recognizable to the skilled artisan. An analog is defined as a substitution of an atom or functional group in the heparin molecule with a different atom or functional group that usually has similar properties. A derivative is defined as an O-desulfated heparin that has another molecule or atom attached to it.

In certain embodiments, an analog of O-desulfated heparin, as described herein, includes compounds having the same functions as O-desulfated heparin for use in the methods of the invention (including minimal anticoagulant activity), and specifically includes homologs that retain these functions. For example, various substituents on the heparin polymer can be removed or altered by any of many means known to those skilled in the art, such as acetylation, deacetylation, decarboxylation, oxidation, etc., so long as such alteration or removal does not substantially increase the low anticoagulation activity of the O-desulfated heparin. Any analog can be readily assessed for these activities by known methods given the teachings herein.

The O-desulfated heparin of the invention may particularly include O-desulfated heparin having modifications, such as reduced molecular weight or acetylation, deacetylation, oxidation, and decarboxylation, as long as it retains its ability to function according to the methods of the invention. Such modifications can be made either prior to or after partial desulfation and methods for modification are standard in the art. As noted above, the O-desulfated heparin can particularly be modified to have a reduced molecular weight, and several low molecular weight modifications of heparin have been developed (see page 581, Table 27.1 Heparin, Lane & Lindall).

Periodate oxidation (described in U.S. Pat. No. 5,250,519, which is incorporated herein by reference) is one example of a known oxidation method that produces an oxidized heparin having reduced anticoagulant activity that may be used according to the present invention. Other oxidation methods known in the art also can be used. Additionally, for example, decarboxylation of heparin is also known to decrease anticoagulant activity, and such methods are standard in the art. Furthermore, some low molecular weight heparins are known in the art to have decreased anti-coagulant activity, including Vasoflux, a low molecular weight heparin produced by a method comprising depolymerization using nitrous acid, followed by periodate oxidation (see, Weitz J I, Young E, Johnston M, Stafford A R, Fredenburgh J C, Hirsh J. Circulation. 99:682-689, 1999).

Modified O-desulfated heparin (or heparin analogs or derivatives) contemplated for use in the present invention can include, for example, periodate-oxidized O-desulfated heparin, decarboxylated O-desulfated heparin, acetylated O-desulfated heparin, deacetylated O-desulfated heparin, deacetylated, oxidized O-desulfated heparin, and low molecular weight O-desulfated heparin. Of course, this is only an example of the heparin analogs or derivatives that could be used. Heparin that is 2-O, 3-O desulfated with an average molecular weight of about 8,000 to about 12,500 Da can be particularly useful according to certain embodiments of the present invention for treating or preventing acute exacerbations of COPD.

The O-desulfated heparin used according to the present invention can be in any form useful for delivery to a patient provided the O-desulfated heparin maintains the activity useful in the methods of the invention, particularly the low anticoagulation activity of the O-desulfated heparin. Non-limiting examples of further forms the O-desulfated heparin may take on that are encompassed by the invention include esters, amides, salts, solvates, prodrugs, or metabolites. Such further forms may be prepared according to any methods that are known in the art, such as, for example, those methods described by J. March, Advanced Organic Chemistry: Reactions, Mechanisms and Structure, 4^{th} Ed. (New York: Wiley-Interscience, 1992), which is incorporated herein by reference.

In the case of solid compositions, it is understood that the compounds used in the methods of the present invention may exist in different forms. For example, the compounds may exist in stable and metastable crystalline forms and isotropic and amorphous forms, all of which are intended to be within the scope of the present invention.

### IV. Pharmaceutical Compositions

While it is possible for the O-desulfated heparin used in the methods of the present invention to be administered in the raw chemical form, it is preferred for the compounds to be delivered as a pharmaceutical composition. Accordingly, there are provided by the present invention pharmaceutical compositions comprising O-desulfated heparin. As such, the compositions used in the methods of the present invention comprise O-desulfated heparin or pharmaceutically acceptable variants thereof.

The O-desulfated heparin can be prepared and delivered together with one or more pharmaceutically acceptable carriers therefore, and optionally, other therapeutic ingredients. Carriers should be acceptable in that they are compatible with any other ingredients of the composition and not harmful to the recipient thereof. Such carriers are known in the art. See, Wang et al. (1980) J. Parent. Drug Assn. 34(6):452-462, herein incorporated by reference in its entirety.

Compositions for use according to the present invention may include short-term, rapid-onset, rapid-offset, controlled release, sustained release, delayed release, and pulsatile release compositions, providing the compositions achieve administration of a compound as described herein. See Remington's Pharmaceutical Sciences (18^{th} ed.; Mack Publishing Company, Eaton, Pa., 1990), herein incorporated by reference in its entirety.

Pharmaceutical compositions for use in the methods of the invention are suitable for various modes of delivery, including oral, parenteral, and topical (including dermal, buccal, and sublingual) administration. Administration can also be via nasal spray, surgical implant, internal surgical paint, infusion pump, or other delivery device. The most useful and/or beneficial mode of administration can vary, especially depending upon the condition of the recipient.

In preferred embodiments, the compositions of the invention are administered intravenously, subcutaneously, or by inhalation (for example, as an aerosol or a micronized dry powder). Particularly preferred modes of delivery include parenteral infusions (such as intravenous and subcutaneous infusions) or periodic injections (including intravenous and subcutaneous periodic injections from once up to four times daily). Administration can also be via inhalation into the lungs as an aerosol in isotonic NaCl, or as a dry powder.

The pharmaceutical compositions of the invention may be conveniently made available in a unit dosage form, whereby such compositions may be prepared by any of the methods generally known in the pharmaceutical arts. Generally speaking, such methods of preparation comprise combining (by various methods) the O-desulfated heparin with a suitable carrier or other adjuvant, which may consist of one or more ingredients. The O-desulfated heparin combined with the one or more adjuvants is then physically treated to present the composition in a suitable form for delivery (e.g., an aqueous suspension).

Compositions for parenteral administration include aqueous and non-aqueous sterile injection solutions, which may further contain additional agents, such as anti-oxidants, buffers, bacteriostats, and solutes, which render the compositions isotonic with the blood of the intended recipient. The compositions may include aqueous and non-aqueous sterile suspensions, which can comprise suspending agents and/or thickening agents. Such compositions for parenteral administration may be presented in unit-dose or multi-dose containers, such as, for example, sealed ampoules and vials, and may be stored in a freeze-dried (lyophilized) condition requiring only the addition of the sterile liquid carrier, for example, water (for injection), immediately prior to use. Extemporaneous injection solutions and suspensions may be prepared from sterile powders, granules, and the like.

In specific embodiments, a patient suffering a COPD exacerbation can be treated with 2-O, 3-O desulfated heparin produced according to methods outlined in U.S. Pat. No. 5,990,097, which is incorporated herein by reference. In certain embodiments, treatment can be effected by administering an intravenous bolus comprising O-desulfated heparin. Such composition can be formed according to various pharmaceutical methods, as discussed herein. Preferably, the bolus is isotonic and has a pH that is neutral to slightly acidic. In a specific embodiment, an intravenous bolus for administration to a patient suffering a COPD exacerbation comprises a 50 mg/ml formulation of 2-O, 3-O desulfated heparin in water with sufficient NaCl added to make the solution isotonic at about 260 to 320 mOsm/ml. The formulation preferably has a pH of about 5 to 7.5. This formulation can be packaged (such as in sterile 20 ml glass vials) and stored at room temperature under low light conditions.

Of course, other solution concentrations could also be used, and a skilled person would recognize a suitable concentration for achieving the desired delivery of O-desulfated heparin in the desired amount of time. For example, an intravenous bolus could comprise O-desulfated heparin in a range of about 5 mg/ml to about 250 mg/ml, about 10 mg/ml to about 200 mg/ml, about 15 mg/ml to about 150 mg/ml, about 20 mg/ml to about 100 mg/ml, or about 25 mg/ml to about 75 mg/ml.

In one embodiment, a patient is treated by administering a first intravenous bolus of O-desulfated heparin at doses ranging from 4 to 8 mg/kg, the drug being dissolved in 50 to 100 ml of 5% dextrose in water or 0.9% NaCl. This bolus dose can be followed by a constantly infused dose for up to 96 hours. In specific embodiments, the constantly infused dose is in the range of 0.35 to 0.6 mg/kg/hr. The infused drug can also be diluted in 5% dextrose in water or 0.9% NaCl for infusion.

When treating a patient suffering an acute exacerbation of COPD using such a method, the amount of O-desulfated heparin used in the bolus and the composition for infusion can vary. The bolus can comprise O-desulfated heparin in an amount of about 0.1 mg/kg of patient body weight to about 20 mg/kg of patient body weight. In further embodiments, the bolus can comprise O-desulfated heparin in an amount of about 0.5 mg/kg to about 18 mg/kg, about 1 mg/kg to about 15 mg/kg, about 2 mg/kg to about 12 mg/kg, or about 3 mg/kg to about 10 mg/kg.

In other embodiments, the constantly infused dose can comprise O-desulfated heparin in an amount providing for delivery of about 0.05 mg per kg of body weight per hour of delivery (mg/kg/hr) to about 5 mg/kg/hr. In still further embodiments, O-desulfated heparin can be constantly infused at a rate of about 0.1 mg/kg/hr to about 3 mg/kg/hr, about 0.15 mg/kg/hr to about 2 mg/kg/hr, about 0.2 mg/kg/hr to about 1 mg/kg/hr, about 0.25 mg/kg/hr to about 0.8 mg/kg/hr, 0.275 mg/kg/hr to about 0.75 mg/kg/hr, or O-desulfated heparin can be constantly infused at a rate of about 0.3 mg/kg/hr to about 0.7 mg/kg/hr.

Likewise, the duration of the constant infusion can also vary. For example, the constant infusion can be carried out for a time of up to about 168 hours. In further embodiments, the constant infusion can be carried out for a time of about 12 hours to about 168 hours, about 18 hours to about 144 hours, about 24 hours to about 120 hours, about 36 hours to about 96 hours, about 48 hours to about 96 hours, or about 60 hours to about 96 hours. Of course, the duration of the constant infusion could vary based on the concentration of the O-desulfated heparin in the infused formulation. It is also understood that the treatment by constant infusion as described herein can be carried out in combination with administration of a bolus, as disclosed above, or could a stand-alone treatment (i.e., carried out without prior administration of a bolus dose. Preferably, constant infusion is carried out for a time sufficient to treat the COPD exacerbation. In certain embodiments (although not required according to the invention), a patient receiving a constant infusion of O-desulfated heparin is hospitalized for the COPD exacerbation. In such embodiments, it is preferable that the constant infusion be carried out until the exacerbation has been reduced or eliminated such that the patient is discharged from the hospital.

Tables 2-5 below illustrate the treatment of a patient suffering an acute exacerbation of COPD by administering a bolus of 8 mg/kg followed by infusion of 0.375 mg/kg/hr for 96 hours. For each bolus dose, a total of 50 mL of solution can be infused. In order to provide additional solution for priming infusion lines, a total of 75 L can be prepared. For example, for a 70 kg subject receiving a bolus does of 8 mg/kg, Table 2 describes the amount of 2-O, 3-O desulfated heparin (referred to as ODSH), the diluent required, and the final solution concentrations for this specific, exemplary bolus dosing scheme.

TABLE 2

| Parameter | Amount | |
|---|---|---|
| Infusion bag volume | 100 | (mL) |
| Delivered volume | 50 | (mL) |
| Total prepared volume | 75 | (mL) |
| Patient weight | 70 | (kg) |
| ODSH bolus dose | 8.0 | (mg/kg) |
| Infusion rate | 200 | (mL/hr) |
| Concentration delivered | 11.2 | (mg/mL) |
| Total volume ODSH added to bag | 16.8 | (mL) |
| Total volume saline added to bag | 58.2 | (mL) |

Table 3 illustrates further exemplary formulations for bolus dosing based on patient weight. The bolus doses can be prepared by combining the calculated amounts of 2-O, 3-O desulfated heparin and 0.9% sodium chloride (i.e., normal saline), or other suitable infusion medium, in a sterile infusion bag. An intravenous infusion line can then be attached to the infusion bag, and the infusion set primed with solution. A Luer lock can be placed at the end of the set. Because 2-O, 3-O desulfated heparin doses are weight based, the amount of 2-O, 3-O desulfated heparin and diluent will both vary by subject weight. The examples of Table 3 are based on an infusion bag volume of 100 mL, a delivered volume of 50 mL, a total prepared volume of 75 mL, a bolus dose of 8 mg/kg, an infusion rate of 200 mL/hr, and an infusion duration of 0.25 hours.

TABLE 3

| Body Weight | ODSH Volume | Saline Volume | ODSH Conc. | Dose |
|---|---|---|---|---|
| (kg) | (mL) | (mL) | (mg/mL) | (mg/kg) |
| 45.0 | 10.8 | 64.2 | 7.20 | 8.0 |
| 47.5 | 11.4 | 63.6 | 7.60 | 8.0 |
| 50.0 | 12.0 | 63.0 | 8.00 | 8.0 |
| 52.5 | 12.6 | 62.4 | 8.40 | 8.0 |
| 55.0 | 13.2 | 61.8 | 8.80 | 8.0 |
| 57.5 | 13.8 | 61.2 | 9.20 | 8.0 |
| 60.0 | 14.4 | 60.6 | 9.60 | 8.0 |
| 62.5 | 15.0 | 60.0 | 10.00 | 8.0 |
| 65.0 | 15.6 | 59.4 | 10.40 | 8.0 |
| 67.5 | 16.2 | 58.8 | 10.80 | 8.0 |
| 70.0 | 16.8 | 58.2 | 11.20 | 8.0 |
| 72.5 | 17.4 | 57.6 | 11.60 | 8.0 |
| 75.0 | 18.0 | 57.0 | 12.00 | 8.0 |
| 77.5 | 18.6 | 56.4 | 12.40 | 8.0 |
| 80.0 | 19.2 | 55.8 | 12.80 | 8.0 |
| 82.5 | 19.8 | 55.2 | 13.20 | 8.0 |
| 85.0 | 20.4 | 54.6 | 13.60 | 8.0 |
| 87.5 | 21.0 | 54.0 | 14.00 | 8.0 |
| 90.0 | 21.6 | 53.4 | 14.40 | 8.0 |
| 92.5 | 22.2 | 52.8 | 14.80 | 8.0 |
| 95.0 | 22.8 | 52.2 | 15.20 | 8.0 |
| 97.5 | 23.4 | 51.6 | 15.60 | 8.0 |
| 100.0 | 24.0 | 51.0 | 16.00 | 8.0 |
| 102.5 | 24.6 | 50.4 | 16.40 | 8.0 |
| 105.0 | 25.2 | 49.8 | 16.80 | 8.0 |
| 107.5 | 25.8 | 49.2 | 17.20 | 8.0 |
| 110.0 | 26.4 | 48.6 | 17.60 | 8.0 |
| 112.5 | 27.0 | 48.0 | 18.00 | 8.0 |
| 115.0 | 27.6 | 47.4 | 18.40 | 8.0 |
| 117.5 | 28.2 | 46.8 | 18.80 | 8.0 |
| 120.0 | 28.8 | 46.2 | 19.20 | 8.0 |
| 122.5 | 29.4 | 45.6 | 19.60 | 8.0 |
| 125.0 | 30.0 | 45.0 | 20.00 | 8.0 |
| 127.5 | 30.6 | 44.4 | 20.40 | 8.0 |
| 130.0 | 31.2 | 43.8 | 20.80 | 8.0 |

For each continuous infusion dose, in certain embodiments, a total of 300 mL of diluted O-desulfated heparin can be prepared. The initial infusion rate can be 10 mL/hr, and the infusion rate may change depending upon aPTT values. For each subject with COPD exacerbation, continuous infusions are preferably prepared at a concentration based upon patient body weight (i.e., the body weight measured within 36 hours of infusion start). Infusion lines are preferentially primed with active drug product. Preferentially, the O-desulfated heparin is maintained in refrigerated conditions (e.g., in the range of 2-8° C.) until used. The infusion solution should be allowed to reach room temperature prior to administration. For example, for a 70 kg subject receiving a continuous infusion of 0.375 mg/kg/hr, Table 4 below describes the amount of O-desulfated heparin and saline required, as well as the final solution concentration.

TABLE 4

| Parameter | Amount | |
|---|---|---|
| Delivered volume | 240 | (mL) |
| Total prepared volume | 300 | (mL) |
| Patient weight | 70 | (kg) |
| ODSH dose | 9.0 | (mg/kg/24 hr) |
| ODSH dose | 0.375 | (mg/kg/hr) |
| ODSH dose | 630 | (mg/24 hr) |
| Infusion rate | 10 | (mL/hr) |
| Volume of saline added to bag | 284.3 | (mL) |
| Volume ODSH delivered in 24 hr | 1 | (mL) |
| Concentration delivered | 2.63 | (mg/mL) |
| Total volume ODSH added to bag | 15.8 | (mL) |

Table 5 below illustrates further exemplary formulations for continuous dosing based on patient weight. The continuous doses can be prepared by combining the calculated amounts of O-desulfated heparin and suitable infusion medium (e.g., 0.9% sodium chloride) in a sterile infusion bag. An intravenous infusion line can then be attached to the infusion bag, and the infusion set primed with drug solution. A Luer lock can then be placed at the end of the set. Because O-desulfated heparin doses are weight based, the amount of O-desulfated heparin and saline will both vary by subject weight. Table 5 below can particularly be useful for calculating the correct parameters for a continuous infusion dose of 0.375 mg/kg/hr.

TABLE 5

| | ODSH | ODSH | | | Conc. |
|---|---|---|---|---|---|
| Body | Dose | Dose | ODSH | Saline | ODSH |
| Weight | (mg/kg/24 | (mg/24 | Vol. | Vol. | Delivered |
| (kg) | hr) | hr) | (mL) | (mL) | (mg/mL) |
| 45.0 | 9.00 | 405.0 | 10.1 | 289.9 | 1.69 |
| 47.5 | 9.00 | 427.5 | 10.7 | 289.3 | 1.78 |
| 50.0 | 9.00 | 450.0 | 11.3 | 288.8 | 1.88 |
| 52.5 | 9.00 | 472.5 | 11.8 | 288.2 | 1.97 |
| 55.0 | 9.00 | 495.0 | 12.4 | 287.6 | 2.06 |
| 57.5 | 9.00 | 517.5 | 12.9 | 287.1 | 2.16 |
| 60.0 | 9.00 | 540.0 | 13.5 | 286.5 | 2.25 |
| 62.5 | 9.00 | 562.5 | 14.1 | 285.9 | 2.34 |
| 65.0 | 9.00 | 585.0 | 14.6 | 285.4 | 2.44 |
| 67.5 | 9.00 | 607.5 | 15.2 | 284.8 | 2.53 |
| 70.0 | 9.00 | 630.0 | 15.8 | 284.3 | 2.63 |
| 72.5 | 9.00 | 652.5 | 16.3 | 283.7 | 2.72 |
| 75.0 | 9.00 | 675.0 | 16.9 | 283.1 | 2.81 |
| 77.5 | 9.00 | 697.5 | 17.4 | 282.6 | 2.91 |
| 80.0 | 9.00 | 720.0 | 18.0 | 282.0 | 3.00 |
| 82.5 | 9.00 | 742.5 | 18.6 | 281.4 | 3.09 |
| 85.0 | 9.00 | 765.0 | 19.1 | 280.9 | 3.19 |
| 87.5 | 9.00 | 787.5 | 19.7 | 280.3 | 3.28 |
| 90.0 | 9.00 | 810.0 | 20.3 | 279.8 | 3.38 |
| 92.5 | 9.00 | 832.5 | 20.8 | 279.2 | 3.47 |
| 95.0 | 9.00 | 855.0 | 21.4 | 278.6 | 3.56 |
| 97.5 | 9.00 | 877.5 | 21.9 | 278.1 | 3.66 |
| 100.0 | 9.00 | 900.0 | 22.5 | 277.5 | 3.75 |
| 102.5 | 9.00 | 922.5 | 23.1 | 276.9 | 3.84 |
| 105.0 | 9.00 | 945.0 | 23.6 | 276.4 | 3.94 |
| 107.5 | 9.00 | 967.5 | 24.2 | 275.8 | 4.03 |
| 110.0 | 9.00 | 990.0 | 24.8 | 275.3 | 4.13 |
| 112.5 | 9.00 | 1012.5 | 25.3 | 274.7 | 4.22 |
| 115.0 | 9.00 | 1035.0 | 25.9 | 274.1 | 4.31 |
| 117.5 | 9.00 | 1057.5 | 26.4 | 273.6 | 4.41 |
| 120.0 | 9.00 | 1080.0 | 27.0 | 273.0 | 4.50 |
| 122.5 | 9.00 | 1102.5 | 27.6 | 272.4 | 4.59 |
| 125.0 | 9.00 | 1125.0 | 28.1 | 271.9 | 4.69 |
| 127.5 | 9.00 | 1147.5 | 28.7 | 271.3 | 4.78 |
| 130.0 | 9.00 | 1170.0 | 29.3 | 270.8 | 4.88 |

Treatment of a patient with COPD exacerbation using a bolus dose of 2-O, 3-O desulfated heparin followed by a constant infusion dose is particularly beneficial in that it will not cause anticoagulation of the blood or a fall in platelets. In certain embodiments, the O-desulfated heparin treatment can be administered in conjunction with antibiotics, corticosteroids, bronchodilators, and, if needed, non-invasive mask ventilation. In most subjects treated with these doses in this manner along with conventional therapy, the patient will experience sufficient improvement in the COPD exacerbation symptoms (e.g., dyspnea, cough, wheezing and sputum production) to allow discharge to home within about 4 days from the bolus dose of drug.

In another preferred embodiment, desulfated heparin is administered via subcutaneous route. With such administration, the drug may be formulated in concentrations suitable for subcutaneous administration. For example, in certain embodiments, a formulation for subcutaneous administration can comprise O-desulfated heparin in a concentration of about 5 mg/ml to about 500 mg/ml, about 10 mg/ml to about 450 mg/ml, about 15 mg/ml to about 400 mg/ml, about 20 mg/ml to about 350 mg/ml, about 25 mg/ml to about 325 mg/ml, about 30 mg/ml to about 300 mg/ml, about 35 mg/ml to about 275 mg/ml, about 40 mg/ml to about 250 mg/ml, about 45 mg/ml to about 225 mg/ml, or about 50 mg/ml to about 200 mg/ml.

The desired amount of O-desulfated heparin can be combined with a suitable medium, such as isotonic saline or sterile water, and injected via the desired method. For example, the formulation could be injected periodically in volumes up to about 2.0 ml subcutaneously.

Alternately, the formulation can be constantly infused into the subcutaneous space, such as through a small gauge butterfly needle (e.g., a 21 to 23 gauge needle). In still further embodiments, a subcutaneous soft catheter of the variety used for insulin infusion can be used to constantly infuse drug subcutaneously. This catheter is conveniently placed into the subcutaneous space of the anterior abdominal wall. A particularly useful catheter for this purpose is the SOF-SET QR®, which can be purchased from the Medtronic Corporation in Northridge, Calif. This catheter is particularly advantageous because it allows for self-placement by patients.

In one embodiment, once the catheter or butterfly needle is placed, the patient can receive a constant infusion of drug by loading an appropriate amount of a formulation (e.g., about 50 mg/ml) into a syringe. The syringe is then placed into the carriage of a mechanical infusion pump, such as the FREEDOM60® infusion pump available from RMS Medical Products in Chester, N.Y. Connected to an indwelling subcutaneous infusion catheter, this pump-catheter infusion system will infuse O-desulfated heparin at a stable, constant rate for up to 72 hours at infusion rates as high as 0.55 mg/kg/hr.

Alternately, the drug formulation can be diluted similarly to that outlined above for continuous intravenous infusion and administered by continuous subcutaneous infusion using a CADD® infusion pump manufactured by Smith Medical International, Colonial Way, Watford, UK.

Drug formulations to treat or prevent COPD exacerbation can also be delivered directly into the respiratory system by inhalation. As an example, a formulation containing 2-O, 3-O desulfated heparin or other suitable heparin can be made in a suitable concentration with additional saline added to render the formulation isotonic at approximately 280 to 320 mOsm/ml. The O-desulfated heparin is preferably provided in the formulation at a concentration similar to formulations for subcutaneous administration. For example, in one embodiment, the formulation for inhalation can comprise O-desulfated heparin in an amount of about 50 to about 200 mg/ml.

A suitable amount of this solution can be placed into the reservoir of a nebulizer, such as a PARI LC® nebulizer and compressor system, available from PARI Innovative Manufacturers, Midlothian, Va. Of course, the amount of solution placed in the nebulizer will vary according to manufacturer's suggestion for the particular nebulizer. This solution can be inhaled from once up to four times daily to deliver 2-O, 3-O desulfated heparin or another suitable heparin directly to the lung and prevent or treat COPD exacerbation in a patient.

In certain embodiments, the compounds and compositions disclosed herein can be delivered via a medical device. Such delivery can generally be via any insertable or implantable medical device, including, but not limited to stents, catheters, balloon catheters, shunts, or coils. In one embodiment, the present invention provides medical devices, such as stents, the surface of which is coated with a compound or composition as described herein. The medical device of this invention can be used, for example, in any application for treating, preventing, or otherwise affecting the course of a disease or condition, such as those disclosed herein.

In another embodiment of the invention, pharmaceutical compositions comprising O-desulfated heparin are administered intermittently. Administration of the therapeutically effective dose may be achieved in a continuous manner, as for example with a sustained-release composition, or it may be achieved according to a desired daily dosage regimen, as for example with one, two, three, or more administrations per day. By “time period of discontinuance” is intended a discontinuing of the continuous sustained-released or daily administration of the composition. The time period of discontinuance may be longer or shorter than the period of continuous sustained-release or daily administration. During the time period of discontinuance, the level of the components of the composition in the relevant tissue is substantially below the maximum level obtained during the treatment.

The preferred length of the discontinuance period depends on the concentration of the effective dose and the form of composition used. The discontinuance period can be at least 2 days, at least 4 days or at least 1 week. In other embodiments, the period of discontinuance is at least 1 month, 2 months, 3 months, 4 months or greater. When a sustained-release composition is used, the discontinuance period must be extended to account for the greater residence time of the composition in the body. Alternatively, the frequency of administration of the effective dose of the sustained-release composition can be decreased accordingly. An intermittent schedule of administration of a composition of the invention can continue until the desired therapeutic effect, and ultimately treatment of the disease or disorder, is achieved.

Administration of the composition can comprise administering O-desulfated heparin in combination with one or more further pharmaceutically active agents (i.e., co-administration). Accordingly, it is recognized that the pharmaceutically active agents described herein can be administered in a fixed combination (i.e., a single pharmaceutical composition that contains both active agents). Alternatively, the pharmaceutically active agents may be administered simultaneously (i.e., separate compositions administered at the same time). In another embodiment, the pharmaceutically active agents are administered sequentially (i.e., administration of one or more pharmaceutically active agents followed by separate administration or one or more pharmaceutically active agents). One of skill in the art will recognized that the most preferred method of administration will allow the desired therapeutic effect.

Delivery of a therapeutically effective amount of a composition according to the invention may be obtained via administration of a therapeutically effective dose of the composition. Accordingly, in one embodiment, a therapeutically effective amount is an amount effective to treat an acute exacerbation of COPD. In another embodiment, a therapeutically effective amount is an amount effective to treat a symptom of an acute exacerbation of COPD. In yet another embodiment, a therapeutically effective amount is an amount effective to prevent the onset of a symptom associated with an exacerbation of COPD.

The concentration of O-desulfated heparin in the composition will depend on absorption, inactivation, and excretion rates of the O-desulfated heparin as well as other factors known to those of skill in the art. It is to be noted that dosage values will also vary with the severity of the condition to be alleviated. It is to be further understood that for any particular subject, specific dosage regimens should be adjusted over time according to the individual need and the professional judgment of the person administering or supervising the administration of the compositions, and that the dosage ranges set forth herein are exemplary only and are not intended to limit the scope or practice of the presently claimed composition. The active ingredient may be administered at once, or it may be divided into a number of smaller doses to be administered at varying intervals of time.

It is contemplated that compositions of the invention comprising one or more active agents described herein will be administered in therapeutically effective amounts to a mammal, preferably a human. An effective dose of a compound or composition for treatment of any of the conditions or diseases described herein can be readily determined by the use of conventional techniques and by observing results obtained under analogous circumstances.

The effective amount of the compositions would be expected to vary according to the weight, sex, age, and medical history of the subject. Of course, other factors could also influence the effective amount of the composition to be delivered, including, but not limited to, the specific disease involved, the degree of involvement or the severity of the disease, the response of the individual patient, the particular compound administered, the mode of administration, the bioavailability characteristics of the preparation administered, the dose regimen selected, and the use of concomitant medication. The compound is preferentially administered for a sufficient time period to alleviate the undesired symptoms and the clinical signs associated with the condition being treated. Methods to determine efficacy and dosage are known to those skilled in the art. See, for example, Isselbacher et al. (1996) Harrison's Principles of Internal Medicine 13 ed., 1814-1882, herein incorporated by reference.

In specific embodiments for intravenous administration, a composition of the invention can be dosed at about 4-12 mg/kg of bodyweight and infused at a rate of about 0.25 to about 0.75 mg/kg/hr. For subcutaneous administration, a patient can be given an initial dose of about 4-12 mg/kg followed by doses of about 6-18 mg/kg subcutaneously every 24 hours in at least two divided doses. For aerosol administration, the composition can be dosed at about 50 to 500 mg (depending on the efficiency of the nebulizer), up to 6 times daily. Of course, the above dosages are intended from purposes of guidance and are not intended to limit the scope of the invention.

In other specific embodiments, treatment bolus doses can range from about 2.0 mg/kg to about 8.0 mg/kg administered intravenously over about 15 minutes or even subcutaneously over about 30 minutes. Constant infusion doses administered intravenously or subcutaneously range from about 0.35 mg/kg/hr to about 0.6 mg/kg/hr for up to about 96 hours. Periodic intravenous or subcutaneous injection doses can particularly comprise a total of about 8 mg/kg to about 16 mg/kg administered intravenously or subcutaneously. The doses can be administered every 24 hours in two to four divided doses for a time of up to 4 days. Doses administered by inhalation can range from about 1 to about 3 ml of formulations containing about 50 to about 200 mg/ml of drug. These doses can be inhaled from once up to four times daily into the lung by nebulizer.

### EXPERIMENTAL

The present invention will now be described with specific reference to various examples. The following examples are not intended to be limiting of the invention and are rather provided as exemplary embodiments.

### Example 1

### Inhibition of P-Selectin-Mediated Attachment of Human Monocytes by 2-O, 3-O Desulfated Heparin

To study the effect of 2-O, 3-O desulfated heparin on P-selectin mediated attachment of inflammatory phagocytes to surfaces, the ability of U937 human monocytes to attach to P-selectin immobilized on plastic microtiter plates was analyzed. U937 cells were used because they demonstrate the same P-selectin dependent vascular rolling as human neutrophils but, unlike neutrophils, can be cultured as a uniform cell line in tissue culture conditions.

High-bind microtiter plastic plates were coated with 8 μg/ml of protein A in 0.2M carbonate-bicarbonate buffer, pH 9.4 (50 μl/well). Plates were washed with phosphate buffered saline containing 1% bovine serum albumin (PBS-BSA) before plates were coated with a P-Selectin-Fc chimera (R&D Systems, Minneapolis, Minn.). Fifty μL of P-Selectin-Fc (1 μg) was added to each well and incubated for 2 hours at room temperature. Following incubation, wells were washed twice with PBS-BSA, and 50 μl of serially diluted 2-O, 3-O desulfated heparin (ODSH) standards (0-1000 g/ml) in 20 mM HEPES buffer containing 125 mM NaCl, 2 mM calcium and 2 mM magnesium was transferred to wells and kept at room temperature for 15 minutes. To the selected set of wells 50 μl of 10 mM EDTA was added to serve as a negative control.

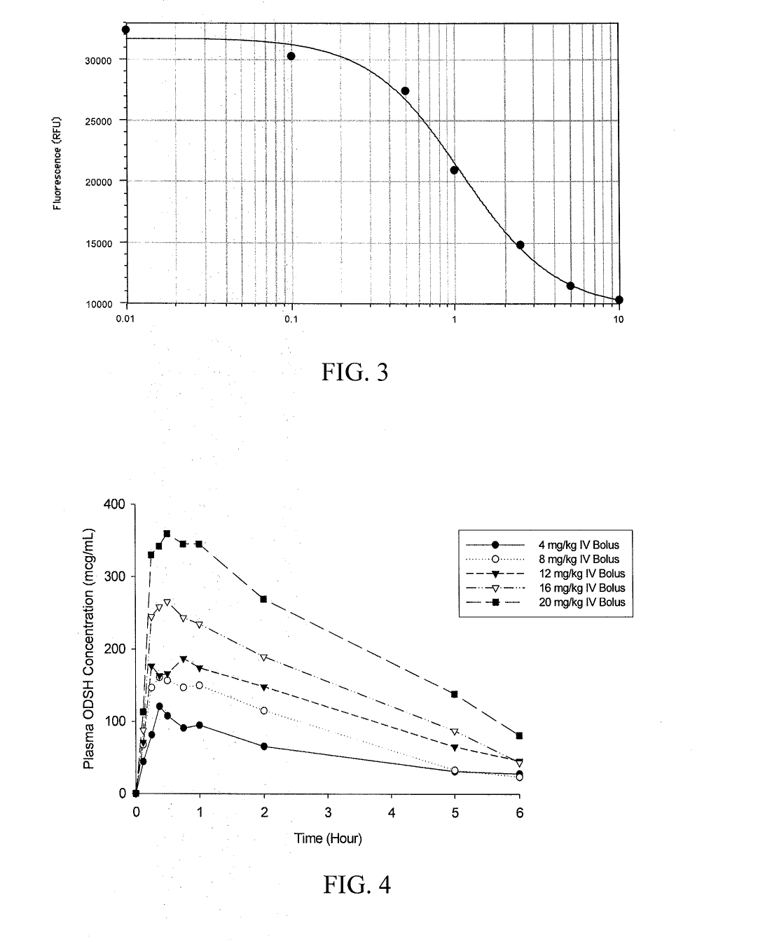

At the end of the incubation period, 50 μl of fluorescent calcien-labeled U937 human monocytes (10⁵ cells/well) were added to the wells containing O-desulfated heparin and EDTA and incubated for 30 min at room temperature. The wells were washed thrice with PBS, the bound cells were lysed with 100 Tris-TritonX-100 buffer, retained calcein cellular fluorescence was measured using excitation of 494 nm and emission of 517 nm. The data is shown in FIG. 3.

The graph in FIG. 3 illustrates that O-desulfated heparin at the concentration of 1 μg/ml prevents 50% binding (IC₅₀) between P-Selectin and U937 cells and ten fold more O-desulfated heparin prevents 90% of binding (IC₉₀), indicating that IC₅₀ and IC₉₀ values for O-desulfated heparin are 1 and 10 μg/ml. Further Examples provided below demonstrate that plasma O-desulfated heparin levels of 25 to 300 μg/ml can be readily and safely attained in humans without deleterious effects. These values are 2.5 to 30 fold higher than the IC₉₀ value of 10 μg/ml, indicating that O-desulfated heparin can abrogate adhesion of inflammatory cells to endothelium in disease conditions such as COPD exacerbation, thereby retarding efflux of inflammatory cells into the diseased lung.

### Example 2

### Safe, Intravenous Bolus Administration of 2-O, 3-O Desulfated Heparin to Humans

A study was performed in 38 volunteer human subjects to assess the effects of escalating bolus doses of 2-O, 3-O desulfated heparin. The study was a Phase I, randomized, double-blind, dose-escalation study with a single-day treatment period. Subjects were between the ages of 18 and 45, were not pregnant, and were normal in body weight. They all had normal coagulation function and hemoglobin values at baseline.

Doses within treatment groups were not escalated, and subjects received a single intravenous dose of O-desulfated heparin over 15 minutes of either active drug or placebo. Two subjects also received an injection of fully anticoagulated unfractionated heparin for comparison. O-desulfated heparin dose groups were run in a series, and safety and tolerance data were evaluated prior to the start of the next dose level (4, 8 12, 16 and 20 mg/kg bolus intravenous doses). Twenty eight (28) subjects randomly received ODSH and 9 subjects were randomized to receive placebo, with an additional two subjects receiving commercially available unfractionated heparin. Dosing was performed according to the schedule shown in Table 6.

TABLE 6

| | | | | | Active | |
|---|---|---|---|---|---|---|
| | | | Active/ | | Agent | |
| | | M:F | Placebo | Active | Dose | Dose |
| Group | n | Ratio | Ratio | Agent | (mg/kg) | (U/kg) |
| 1 | 8 | 4:4 | 3:1 (within | ODSH | 4 or 0 | na |
| | | | gender) | | | |
| 2 | 8 | 8:0 | 3:1 | ODSH | 8 or 0 | na |
| 3 | 8 | 8:0 | 3:1 | ODSH | 12 or 0 | na |
| 4 | 8 | 8:0 | 3:1 | ODSH | 16 or 0 | na |
| 5 | 5 | 5:0 | 4:1 | ODSH | 20 or 0 | na |
| 6 | 2 | 2:0 | 2:0 | Unfractionated | 0.571 | 80 |
| | | | | heparin | | |

1 mg heparin = 140 units

For each bolus dose, O-desulfated heparin as a 50 mg/ml formulation was diluted with normal saline and a total volume of 50 ml was infused over 15 minutes containing the calculated amount of O-desulfated heparin the subject was to receive. Placebo consisted of 50 ml of normal saline infused over 15 minutes. For subjects receiving heparin, 5,000 units (approximately 0.5 mg/kg) of heparin was diluted into 50 ml of normal saline and infused over 15 minutes.

Immediately before infusion and beginning 7 minutes after the start of each infusion, blood was drawn at periodic times for 24 hours to monitor the effect of infusion on the following laboratory studies: activated partial thromboplastin time (aPTT); prothrombin time (PT); activated clotting time (ACT); and O-desulfated heparin plasma level. Serum chemistries and a complete blood count were checked immediately before infusion and at eight (8) and twenty-four (24) hours later. Using values for aPTT and O-desulfated heparin levels, pharmacokinetic parameters were calculated by noncompartmental methods using a commercial software program (PhAST 2.3-001). The following pharmacokinetic parameters were calculated:
a) Maximum measured plasma concentration (Cₘₐₓ);
b) First-order terminal elimination rate constant (Kel), calculated from a semi-log plot of the serum concentration versus time curve; this parameter was calculated by linear least-square regression analysis using the maximum number of points in the terminal log-linear phase (e.g., 3 or more non-zero serum concentrations);
c) Time of the maximum measured drug plasma concentration (tₘₐₓ);
d) The area under the plasma concentration versus time curve from time 0 to the last observation (AUC 0-t), calculated by the linear trapezoidal method;
e) The area under the plasma concentration versus time curve from time 0 to infinity (AUCinf), which was calculated as the sum of AUC 0-t plus the ratio of the last measurable serum concentration to the elimination rate constant;
f) First-order terminal elimination (t_{1/2}), calculated as 0.693/Kel;
g) Total body clearance (CL), calculated as Dose/AUCinf, and
h) Total volume of distribution (Vdss), calculated as MRT×CL.

No serious adverse events were noted and none of the subjects were discontinued from the study due to an adverse event. No treatment- or dose-related trends were noted in the serum chemistry, hematological, urinalysis, or physical exam findings. Specifically, bolus O-desulfated heparin did not increase blood glucose, nor did it elevate blood pressure. Mean ACT value at 15 minutes for the two heparin treated patients receiving about 0.5 mg/kg heparin was 333 seconds; however, the mean ACT value for subjects receiving 20 mg/kg O-desulfated heparin was only 207 seconds (a difference of over 100 seconds, even though the drug dose was 40-fold higher). Thus, O-desulfated heparin is substantially less anticoagulating than unfractionated heparin.

The mean plasma concentrations of O-desulfated heparin for the dose levels studied are presented in FIG. 4. O-desulfated heparin plasma concentrations peaked shortly after the end of infusion and then declined in an exponential manner. Descriptive statistics of the pharmacokinetic parameters of O-desulfated heparin in this study are summarized below in Table 7.

TABLE 7

| | ODSH Dose Levels | | | | |
|---|---|---|---|---|---|
| Pharmokinetic | Group 1 | Group 2 | Group 3 | Group 4 | Group 5 |
| Parameters | 4 mg/kg | 8 mg/kg | 12 mg/kg | 16 mg/kg | 20 mg/kg |

Geometric Mean CV %

| | | | | | |
|---|---|---|---|---|---|
| AUC 0-t | 307.2 | 461.9 | 619.1 | 886.9 | 1322.1 |
| (μg h/mL) | (52.9%) | (46.9%) | (62.3%) | (21.2%) | (7.8%) |
| AUCinf | 415.2 | 629.2 | 1086.5 | 1075.8 | 1638.7 |
| (μg h/mL) | (44.2%)* | (18.2%)** | (19.7%)* | (29.4%) | (6.5%) |
| Cₘₐₓ | 130.76 | 163.74 | 179.28 | 285.38 | 366.73 |
| (μg/mL) | (34.1%) | (19.7%) | (66.8%) | (13.5%) | (9.7%) |

Arithmetic Mean +/− SD

| | | | | | |
|---|---|---|---|---|---|
| t_{1/2} (h) | 2.585 ± 1.1225* | 1.933 ± 0.4537** | 2.724 ± 0.6667* | 2.261 ± 0.8548 | 2.637 ± 0.4765 |
| CL (mL/h/kg) | 10.254 ± 3.8984* | 12.882 ± 2.3521** | 11.202 ± 2.1722* | 15.364 ± 4.0502 | 12.526 ± 0.2090 |
| Vdss (mL/kg) | 34.95 ± 11.679* | 35.13 ± 6.580** | 42.12 ± 3.170* | 47.25 ± 7.944 | 45.56 ± 8.256 |
| MRT (h) | 3.780 ± 1.6710* | 2.775 ± 0.6087** | 3.894 ± 0.9516* | 3.287 ± 1.0560 | 3.639 ± 0.6670 |

Median (Min-Max)

| | | | | | |
|---|---|---|---|---|---|
| tₘₐₓ (h) | 0.47 | 0.37 | 0.88 | 0.50 | 0.50 |
| | (0.25-1.00) | (0.25-0.62) | (0.25-2.00) | (0.37-0.75) | (0.37-1.00) |

*For these parameters n = 4;
**For these parameters n = 5

Mean clearance values of O-desulfated heparin were consistent throughout the dose range studied (values ranged from 10.3 to 15.4 mL/h/kg), indicating a dose proportional increase in pharmacokinetic parameters over the dose range studied in the evaluation. Mean elimination half-life values of O-desulfated heparin from 4 to 20 mg/kg were short, with mean values ranging from 1.93 to 2.72 hours. Median tₘₐₓ values of O-desulfated heparin were observed shortly after the end of the infusion period. Tₘₐₓ values were comparable over the dose range of 4 to 20 mg/kg, with values ranging from 0.37 to 0.88 hours.

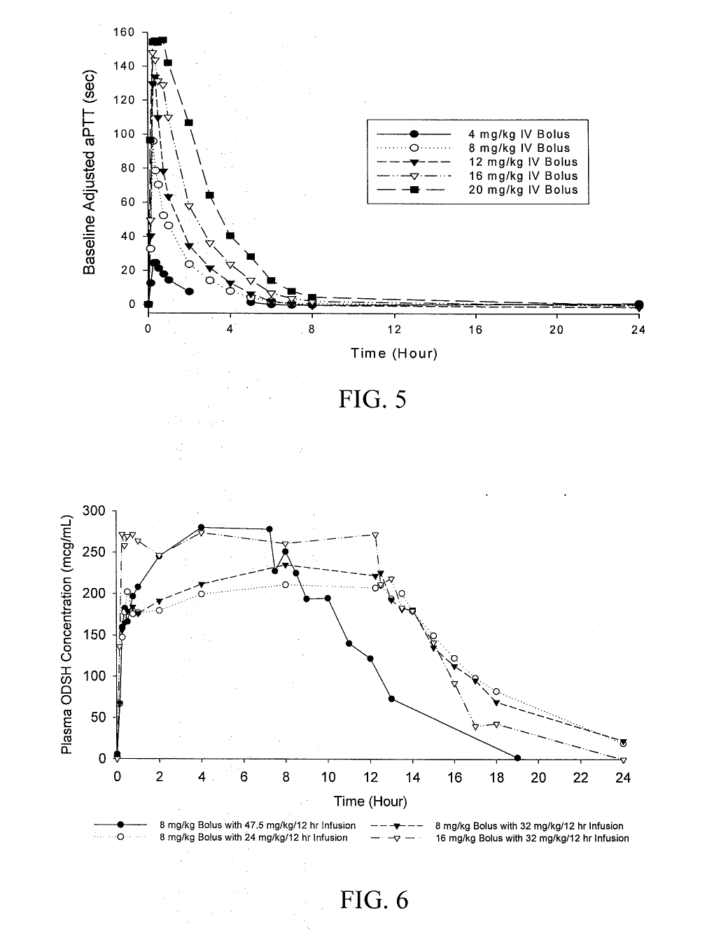

The change from baseline in aPTT is shown in FIG. 5. O-desulfated heparin produced a rapid increase in aPTT over the infusion period in a dose-dependent fashion. PT and ACT also increased in a dose-dependent manner.

Platelet counts for patients treated with O-desulfated heparin and patients treated with a placebo (in all dose groups) are shown below in Table 8, wherein values are provided as thousands/μL blood (mean±SD). Administered O-desulfated heparin did not produce the >50% fall in platelet counts characteristic of heparin-induced thrombocytopenia (HIT). These findings indicate that this heparin analog (O-desulfated heparin) is safe from producing HIT during use at clinical doses in humans.

TABLE 8

| | | Before Bolus Dose | | 24 h After Bolus Dose | |
|---|---|---|---|---|---|
| Dose | | ODSH | Placebo | ODSH | Placebo |
| 4 | mg/kg | 267 ± 72 | 287 ± 84 | 207 ± 70 | 267 ± 73 |
| 8 | mg/kg | 248 ± 39 | 258 ± 30 | 236 ± 34 | 257 ± 8 |
| 12 | mg/kg | 236 ± 63 | 293 ± 82 | 221 ± 52 | 309 ± 91 |
| 16 | mg/kg | 260 ± 37 | 242 ± 47 | 252 ± 37 | 242 ± 71 |
| 20 | mg/kg | 288 ± 27 | 278 | 278 ± 34 | 274 |

These data demonstrate that O-desulfated heparin is safe when administered at large bolus doses, producing O-desulfated heparin plasma levels >300 μg/mL while increasing the ACT much less at the highest dose (20 mg/kg) than even 40-fold lower concentrations of unfractionated heparin administered at doses of approximately 0.5 mg/kg. Used in these bolus doses, O-desulfated heparin also does not elevate blood glucose, increase blood pressure, or produce catastrophic thrombocytopenia characteristic of HIT.

### Example 3

### Safe Intravenous Bolus Administration and 12 Hour Infusion of 2-O, 3-O Desulfated Heparin to Normal Humans

A study was performed in twenty-four (24) healthy adult subjects to assess the effects of a bolus dose and 12 hour infusion of 2-O, 3-O desulfated heparin. The study was a Phase I, randomized, double-blind, dose escalation study with single-day treatment periods. Subjects were males between the ages of 18 and 45, and were normal in body weight. They all had normal coagulation function and hemoglobin values at baseline. Doses within treatment group were not escalated, and subjects received either active drug (O-desulfated heparin) or placebo treatment. Eighteen (18) subjects were randomized to receive O-desulfated heparin and six (6) subjects were randomized to receive placebo. Subjects received either O-desulfated heparin or placebo as described below in Table 9.

TABLE 9

| | | | | Continuous Infusion |
|---|---|---|---|---|
| | | Active/Placebo | Bolus ODSH | ODSH |
| Group | n | Ratio | (mg/kg) | (mg/kg/12 hr) |
| 1 | 2 | 2:0 | 8 | 47.5 |
| 2 | 6 | 4:2 | 8 | 24 |
| 3 | 8 | 6:2 | 8 | 32 |
| 4 | 8 | 6:2 | 16 | 32 |

For each subject, O-desulfated heparin as a 50 mg/ml formulation was diluted with normal saline and administered as a bolus infused over 15 minutes containing the calculated amount of O-desulfated heparin the subject was to receive, followed by a constant infusion for 12 hours of O-desulfated heparin diluted in saline. Placebo consisted of 50 ml of normal saline infused over 15 minutes, followed by normal saline infused for 12 hours.

Immediately before infusion and after the start of each infusion, blood was drawn at periodic times (over a total 24 hour period) to monitor the effect of infusion on the following laboratory studies: activated partial thromboplastin time (aPTT); prothrombin time (PT); activated clotting time (ACT); and O-desulfated heparin plasma level. Serum chemistries and a complete blood count were checked immediately before infusion and again periodically for up to twenty-four (24) hours later.

Using values for aPTT and O-desulfated heparin levels, pharmacokinetic parameters were calculated by noncompartmental methods using a commercial software program (PhAST 2.3-001). The following pharmacokinetic parameters were calculated (using the same definition for each as described above): Cₘₐₓ; Kel; tₘₐₓ; AUC 0-t; AUCinf, t_{1/2}; CL; and Vdss.

No serious adverse events were noted and none of the subjects were discontinued from the study due to an adverse event. No treatment- or dose-related trends were noted in the serum chemistry, hematological, urinalysis, or physical exam findings. Specifically, bolus O-desulfated heparin did not increase blood glucose, nor did it elevate blood pressure. Mean plasma concentrations of O-desulfated heparin for the bolus and infusion doses studied are presented in FIG. 6.

O-desulfated heparin plasma concentrations peaked shortly after the end of bolus infusion in all groups except those subjects who received 47.5 mg/kg over 12 hr (4 mg/kg/hr). These subjects had O-desulfated heparin levels peak at about 275 μg/ml beginning approximately 4 hours after initiation of infusion. In this group, infusions were discontinued at 8 hours because of a rise in aPTT to sustained values greater than 120 seconds.

After discontinuation of the infusion in this group, O-desulfated heparin levels fell exponentially over the next 12 hours, and a similar drop was identified in the remaining three infusion dose groups. Descriptive statistics of the pharmacokinetic parameters of O-desulfated heparin in this study for Group 2 through Group 4 are summarized below in Table 10.

TABLE 10

| | ODSH Dose Levels | | |
|---|---|---|---|
| | Group 2 | Group 3 | Group 4 |
| | 8 mg/kg Bolus with | 8 mg/kg Bolus with | 16 mg/kg Bolus with |
| Pharmacokinetic | 24 mg/kg/12 hr | 32 mg/kg/12 hr | 32 mg/kg/12 hr |
| Parameters | infusion (n = 4) | infusion (n = 6) | infusion (n = 3) |

Geometric Mean CV %

| | | | | | | |
|---|---|---|---|---|---|---|
| AUC 0-t (μg h/mL) | 3,472.4 | 28.4% | 3,639.7 | 19.7% | 3,895.2 | 26.5% |
| AUCinf (μg h/mL) | 3,562.0 | 29.4% | 3,755.5 | 20.7% | 4,633.3 | N/C (n = 2) |
| Cₘₐₓ (μg/mL) | 216.79 | 19.4% | 246.39 | 18.5% | 301.12 | 23.8% |

Arithmetic Mean +/− SD

| | | | | | | |
|---|---|---|---|---|---|---|
| t_{1/2} (h) | 2.602 | 0.9800 | 3.696 | 0.9576 | 1.598 | N/C (n = 2) |
| CL (mL/h/kg) | 9.287 | 2.9419 | 10.835 | 2.1722 | 10.367 | N/C (n = 2) |
| Vdss (mL/kg) | 30.61 | 5.123 | 39.61 | 11.051 | 20.35 | N/C (n = 2) |
| MRT (h) | 3.568 | 1.2710 | 3.702 | 0.8641 | 1.961 | N/C (n = 2) |

Median (Min-Max)

| | | | | | | |
|---|---|---|---|---|---|---|
| tₘₐₓ (h) | 12.38 | (0.75-13.0) | 10.13 | (8.0-12.50) | 0.75 | (0.25-4.0) |

N/C = Not calculated when n < 3

Pharmacokinetic results show that the systemic exposure to O-desulfated heparin was similar following the 3 dosing regimens. Mean clearance values of the 3 dosing regimens were similar, suggesting that the pharmacokinetics of O-desulfated heparin is linear. Mean Cₘₐₓ values were comparable in both groups given the 8 mg/kg bolus (217 vs. 246 μg/mL). On the other hand, Cₘₐₓ values were greater following 16 mg/kg with the 32 mg/kg/12 hour infusion compared to the 8 mg/kg with the 32 mg/kg/12 hour infusion. The observed median tₘₐₓ values decreased from 12.4 to 10.1 hours when the infusion dose was increased 24 to 32 mg/kg/12 hour in the 8 mg/kg bolus regimens. Similarly, tₘₐₓ values deceased from 10.1 to 0.75 hours when the bolus dose was increased from 8 to 16 mg/kg in the 32 mg/kg/12 hour infusion regimens. This increase suggests that the 16 mg/kg loading dose of O-desulfated heparin caused Cₘₐₓ to be reached at an earlier timepoint as compared to the other two treatments provided.

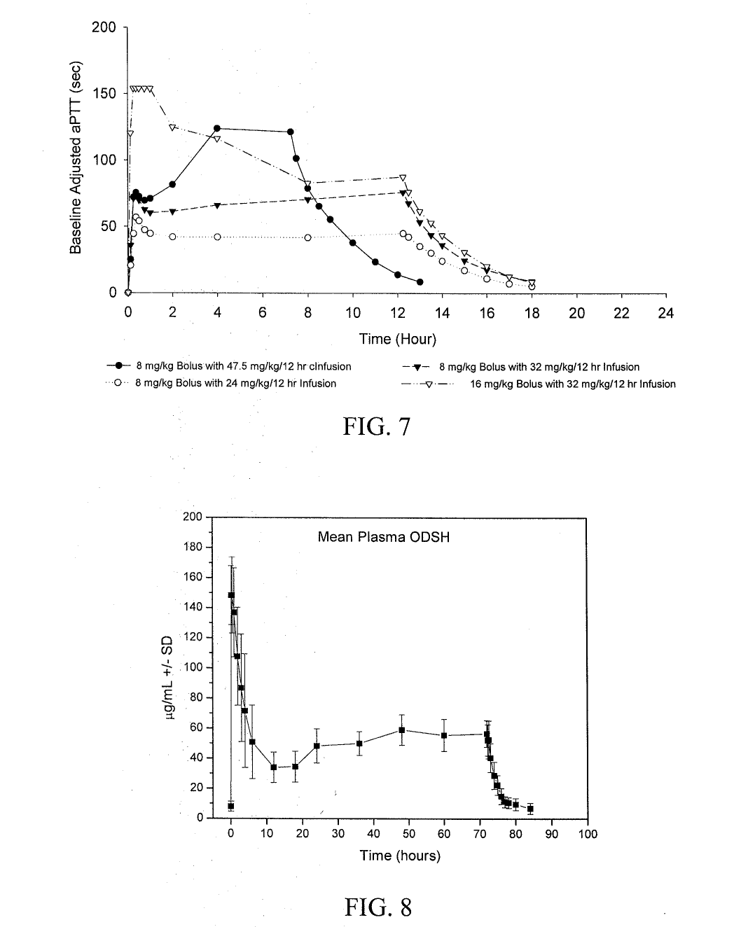

Mean value for aPTT for all groups is summarized in FIG. 7. O-desulfated heparin bolus and infusion at the doses chosen induced sustained increases in aPTT over the 12 hour infusion period. Group 2 receiving a bolus of 8 mg/kg followed by an infusion of 24 mg/kg/12 hours (or 2 mg/kg/hr) experienced an immediate and sustained increase in aPTT of approximately 50 seconds above baseline (or on average an aPTT of about 75 to 80 seconds absolute), indicating that this dose (8 mg/kg bolus followed by 2 mg/kg/hr) would be useful to induce immediate therapeutic anticoagulation in subjects in need of this type of treatment. Subjects in group 1 (8 mg/kg bolus with 47.5 mg/kg/12 hour infusion) did not complete the 12-hour infusion because of a sustained elevation of aPTT of >120 seconds.

Platelet counts for O-desulfated heparin- and placebo-treated patients in all dose groups are shown below in Table 11, wherein platelet values are provided as thousands/μL blood (mean±SD). O-desulfated heparin did not produce the >50% fall in platelet counts characteristic of heparin-induced thrombocytopenia (HIT), indicating that this heparin analog (ODSH) is safe from producing HIT during use at clinical doses in humans.

TABLE 11

| | Before Bolus Dose | | 24 h After Bolus Dose | |
|---|---|---|---|---|
| Dose | ODSH | Placebo | ODSH | Placebo |
| 8 mg/kg bolus with | 285 ± 45 | | 256 ± 30 | |
| 47.5 mg/kg/12 hr | | | | |
| infusion | | | | |
| 8 mg/kg bolus with | 244 ± 40 | 306 ± 77 | 222 ± 39 | 267 ± 64 |
| 24 mg/kg/12 hr | | | | |
| infusion | | | | |
| 8 mg/kg bolus with | 303 ± 63 | 242 ± 17 | 277 ± 62 | 205 ± 52 |
| 32 mg/kg/12 hr | | | | |
| infusion | | | | |
| 16 mg/kg bolus with | 283 ± 50 | 227 ± 44 | 247 ± 43 | 213 ± 51 |
| 32 mg/kg/12 hr | | | | |
| infusion | | | | |

The data provided in Table 11 demonstrate that O-desulfated heparin is safe when administered in large boluses followed by infusion at doses which produce sustained anticoagulation. O-desulfated heparin levels achieved in the dose group receiving a bolus of 8 mg/kg followed by 24 mg/kg/12 hr (2 mg/kg/hr) and therapeutically anticoagulated with an increase in aPTT of about 50 seconds above baseline were sustained at approximately 200 μg/ml plasma. This is 200-fold higher than the IC₅₀ and 20-fold higher than the IC₉₀ concentrations for P-selectin inhibition outlined in Example 1. Therefore, O-desulfated heparin at this dose should be a safe drug for restoring both therapeutic anticoagulation and inhibition of neutrophil and other inflammatory cell egress from the vascular space into the inflamed lung. Used in these bolus and infusion doses to produce therapeutic anticoagulation, O-desulfated heparin also does not elevate blood glucose, increase blood pressure, or produce catastrophic thrombocytopenia characteristic of HIT.

### Example 4

### Safe Intravenous Bolus Administration and 72 Hour Infusion of 2-O, 3-O Desulfated Heparin to Normal Humans

A study was performed in eight (8) healthy adult male and female subjects to assess the effects of a bolus dose and 72 hour infusion of 2-O, 3-O desulfated heparin. The study was a Phase I study with a three day treatment period. Doses were adjusted to maintain an aPTT level of 40-45 seconds. Subjects were between the ages of 18 and 60, were not pregnant, and were normal in body weight. They all had normal coagulation function and hemoglobin values at baseline.

Subjects received an initial bolus of 8 mg/kg of O-desulfated heparin over 15 minutes, followed by 72 hours continuous infusion beginning at 0.58 mg/kg/hr. For each subject O-desulfated heparin as a 50 mg/ml formulation was diluted with normal saline and administered as a bolus infused over 15 minutes containing the calculated amount of O-desulfated heparin the subject was to receive, followed by infusion of O-desulfated heparin diluted in saline. The infusion dose was adjusted to maintain an aPTT of 40-45 seconds. Immediately before infusion and after the start of each infusion, blood was drawn at periodic times (over a total 72 hour period) to monitor the effect of infusion on the following laboratory studies: activated partial thromboplastin time (aPTT), prothrombin time (PT), activated clotting time (ACT), and O-desulfated heparin plasma level. Serum chemistries and a complete blood count were checked immediately before infusion and again periodically for up to 240 hours later. Using values for aPTT and O-desulfated heparin levels, pharmacokinetic parameters were calculated by noncompartmental methods using a commercial software program (PhAST 2.3-001). The following pharmacokinetic parameters were calculated, as described above: Cₘₐₓ; Kel; tₘₐₓ; AUC O-t; AUCinf, t_{1/2}; CL; and Vdss.

No serious adverse events occurred in this study and none of the subjects were discontinued from the study due to an adverse event. Specifically, bolus O-desulfated heparin did not increase blood glucose, nor did it elevate blood pressure. Mild ecchymosis was reported in one subject and was assessed as unlikely to be related to O-desulfated heparin. The infusion in two subjects was not able to be completed because of infusion pump mechanical failure. As commonly observed with therapeutic levels of unfractionated or low molecular weight heparins, transient elevations in serum alanine aminotransferase (ALT) and aspartate aminotransferase (AST) were observed in seven subjects, beginning on the third day of drug administration, peaking at day five or six, and returning to normal within two weeks. Such observations are reported by Dukes GE Jr., et al., Ann Int Med 100:646-650, 1984; and Carlson MK, et al., Pharmacotherapy 21:108-113, 2001.

There was no clear relationship to O-desulfated heparin dose. In no case did ALT or AST rise to greater than seven times the upper limit of normal (ULN). Average peak ALT and AST was 3.1 times ULN. These transient elevations in tranaminases have been well-recognized to occur by regulatory agencies. The phenomenon is thought to be a class effect of all heparinoids and is not believed to be associated with adverse outcomes. Transaminase elevations from heparins were addressed in deliberations of a Canadian government scientific advisory panel on hepatotoxicity of health care products. Heparin was classified as an agent causing transaminasemia without significant liver damage. The Scientific Advisory Panel on Hepatotoxicity noted that heparin frequently causes an increase in transaminases after a few days of treatment but does not cause significant liver damage. The mechanism by which these agents increase transaminases is unknown, but the characteristics suggest a biochemical effect (Scientific Advisory Panel on Hepatotoxicity, Draft recommendations concerning “Recommendations from the Scientific Advisory Panel Sub-groups on Hepatotoxicity: Hepatotoxicity of Health Care Products”. Oct. 15, 2004. Available on-line at http://www.hs-sc.gc.ca/dhp-mps/prodpharma/activit/sci-consult/hepatotox/sap_gcs_hepatotox_{—}2004-07-26_e.html). Transaminase elevations from heparin are also recognized by the U.S. Food and Drug Administration to occur commonly and to not portend risk of serious liver injury (Drug-induced liver injury: premarketing clinical evaluation, Center for Drug Evaluation and Research, U.S. Food and Drug Administration October, 2007. Available at: http://www.fda.gov.cder/guidance/index.htm).

To achieve the goal of maintaining an aPTT of 40-45 seconds, the infusion rate was adjusted upward in all subjects so that subjects were infused with O-desulfated heparin at 0.64 to 1.39 mg/kg/hr. The mean plasma O-desulfated heparin concentrations for subjects is shown in FIG. 8. O-desulfated heparin plasma concentrations near the end of infusion was approximately 50 μg/ml. Descriptive statistics of the pharmacokinetic parameters of O-desulfated heparin in this study are summarized below in Table 12.

TABLE 12

| | |
|---|---|
| | ODSH Dose Levels |
| | 8 mg/kg Bolus with |
| | Infusion for 12 hr, |
| Pharmacokinetic | Final 0.64-1.39 mg/kg/hr |
| Parameters | (n = 6) |

Geometric Mean CV %

| | | |
|---|---|---|
| AUC | 4,053 | 9.9% |
| (μg h/mL) | | |
| Cₘₐₓ | 156 | 15.0% |
| (μg/mL) | | |

Arithmetic Mean +/− SD

| | | |
|---|---|---|
| t_{1/2} (h) | 3.3 | ±1.0 |
| CL (mL/h/kg) | 10.2 | ±1.0 |
| Vdss (mL/kg) | 48.9 | ±16.0 |
| MRT (h) | 2.0 | ±2.8 |

Median (Min-Max)

| | | |
|---|---|---|
| tₘₐₓ (h) | 0.5 | (0.25-0.5) |

Phamacokinetic results showed that mean AUC was 4053 μg hr/mL, with a range of 3,528 to 4,694 μg hr/mL. Mean clearance value (CL) was 10.2 mL/hr/kg with a range from 8.8 to 11.8 mL/h/kg. The mean Cₘₐₓ was 156 μg/mL, with a range of 131 to 192 μg/mL. Mean Vdss was 48.9 mL/kg, with a range of 23.7 to 66.2 mL/kg. Median tₘₐₓ was 0.5 hours, with very little variation in the minimum to maximum range. The mean value of t_{1/2} was 3.3 hours, with a range of 1.9 to 4.4 hours. Mean MRT was 2.0 hours, with a range of −0.9 to 5.93 hours.

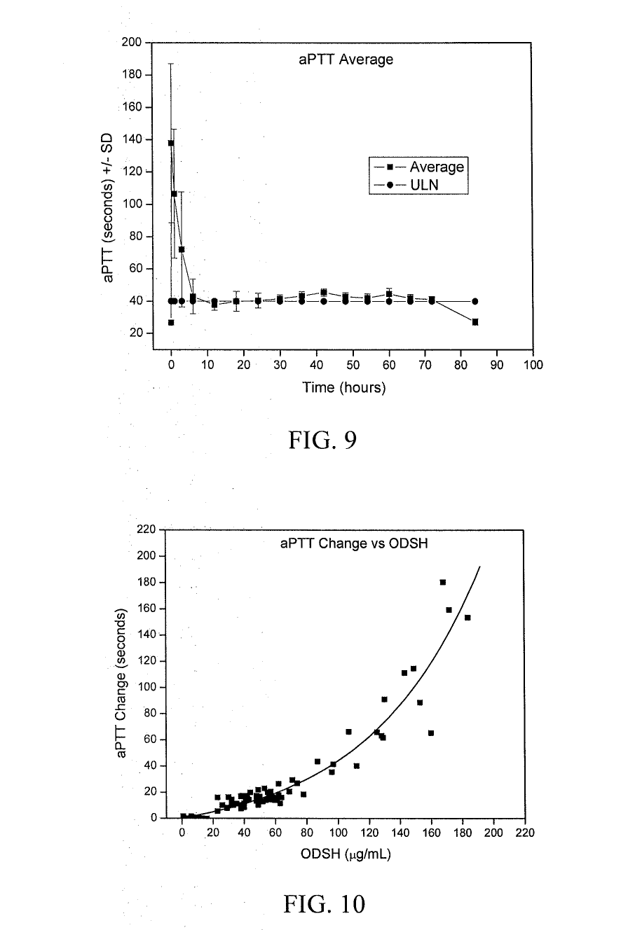

The mean aPTT in subjects over the 72 hours of study is shown in FIG. 9. O-desulfated heparin produced a rapid increase in aPTT over the bolus infusion, but values fell to within the range of 40-45 seconds as the infusion was adjusted. The relationship between change in aPTT from baseline and O-desulfated heparin levels for this study is shown in FIG. 10.

Platelet counts for the O-desulfated heparin-treated subjects are shown below in Table 13. The table shows platelet counts after 8 gm/kg bolus followed by 72 hour infusion to aPTT of 40-45 seconds, with platelet values provided as thousands/μL blood (mean±SD). O-desulfated heparin did not produce the >50% fall in platelet counts characteristic of heparin-induced thrombocytopenia (HIT), indicating that this heparin analog is safe from producing HIT during use at these clinical doses in humans.

TABLE 13

| Day Before | Infusion | Infusion | Day After |
|---|---|---|---|
| Infusion | Day 2 | Day 3 | Infusion |
| 261 ± 40 | 258 ± 39 | 272 ± 42 | 261 ± 37 |

These data demonstrate that O-desulfated heparin is safe when administered at a bolus of 8 mg/kg followed by doses of 0.64 to 1.39 mg/kg/hr for 72 hours to maintain an aPTT of 40-45 seconds, producing sustained plasma O-desulfated heparin levels of approximately 50 μg/ml. This is 50-fold higher than the IC₅₀ and 5-fold higher than the IC₉₀ concentrations for P-selectin inhibition outlined in Example I. Therefore, O-desulfated heparin at this dose should be a safe drug for inhibiting neutrophil and other inflammatory cell egress from the vascular space into the inflamed lung without producing sustained anticoagulation. Used in these bolus and infusion doses to treat or prevent lung inflammation, O-desulfated heparin also does not elevate blood glucose, increase blood pressure, or produce catastrophic thrombocytopenia characteristic of HIT.

### Example 5

### Safe Intravenous Bolus Administration and 72 Hour Infusion of O-Desulfated Heparin to Human Subjects Suffering an Acute Exacerbation of COPD

A study was performed in six volunteer human subjects to assess the benefit of 2-O, 3-O desulfated heparin in reducing lung and systemic inflammation and the shortening duration of illness in acute exacerbations of COPD. The study was a Phase I, open-label study in which subjects received a bolus of 8 mg/kg of intravenous O-desulfated heparin, followed by infusion at a constant dose of 0.5 mg/kg/hr for 72 hours. Subjects consisted of individuals suffering an acute exacerbation of COPD sufficient to require admission to the hospital for treatment of disease. The subjects consisted of individuals who had smoked at least 10 pack years of cigarettes and had an acute exacerbation of COPD characterized by an increase in cough, sputum production, and shortness of breath deemed severe enough by their treating physicians to require hospitalization for care. At study entry, the average forced expiratory volume in one second (FEV₁) in the subjects was 0.78±0.23 liters, and the ratio of forced expiratory volume in one second to forced vital capacity (FEV₁/FVC) in subjects was 48±11%. Subject patients consisted of three (3) men and three (3) women with normal coagulation function. Entry characteristics, including hemoglobin, are listed in Table 14.

TABLE 14

| Subject | Age | | Weight | FEV₁ | FEV₁/FVC | Hemoglobin |
|---|---|---|---|---|---|---|
| ID | (years) | Sex | (kg) | (L) | (%) | (g/dL) |
| 08-001 | 69 | F | 63.5 | 0.71 | 36 | 12.9 |
| 06-001 | 57 | F | 79.5 | 0.75 | 54 | 13.6 |
| 08-002 | 66 | M | 54.3 | 0.75 | 38 | 15.7 |
| 12-001 | 72 | M | 99.5 | 0.52 | 42 | 12.9 |
| 12-002 | 68 | M | 84.1 | 1.21 | 66 | 18.9 |
| 08-003 | 68 | F | 40.4 | 0.72 | 49 | 13.1 |

Subjects were treated with medical regimens decided upon by their individual physicians, and received inhaled bronchodilators, intravenous or oral corticosteroids, and intravenous or oral antibiotics in keeping with the current standards of care for COPD exacerbation (Bach PB, et al., Ann Intern Med 134:600-620, 2001). Subjects received a bolus infusion of 8 mg/kg O-desulfated heparin in 50 ml normal saline over 15 minutes, followed by constant infusion of O-desulfated heparin at 0.5 mg/kg/hr for the next 72 hours, or until hospital discharge (whichever was first). The time when patients were sufficiently improved to allow them to be discharged was a decision left solely to the individual patients' treating physicians. Immediately before O-desulfated heparin infusion (and periodically afterward for the next 4 days and for up to 60 days thereafter), blood was drawn to monitor the effect of infusion on the following parameters: activated partial thromboplastin time (aPTT), O-desulfated heparin levels, serum chemistries (including transaminases), complete blood count, and C-reactive protein (CRP). C-reactive protein was monitored as a measure of systemic and lung inflammation.

No serious adverse events were noted and none of the subjects were discontinued from the study due to an adverse event. Specifically, O-desulfated heparin did not produce elevation of blood pressure. Because all subjects received corticosteroids as part of their therapy, the effect of O-desulfated heparin on blood sugar could not be determined in this group of patients. Only one subject had liver function tests elevated above the normal range, and only on day 5. For this individual, aspartate aminotransferase (AST) was 2.9 times upper limit of normal and alanine aminotransferase (ALT) was 1.5 times the upper limit of normal. As expected, when compared to their individual baselines, most subjects had transient increases in AST and ALT as is characteristic for subjects treated with heparins. AST and AST were normal in all subjects when tested two weeks after hospital admission. Nine mild and three moderate adverse events were noted but none were deemed related to O-desulfated heparin.

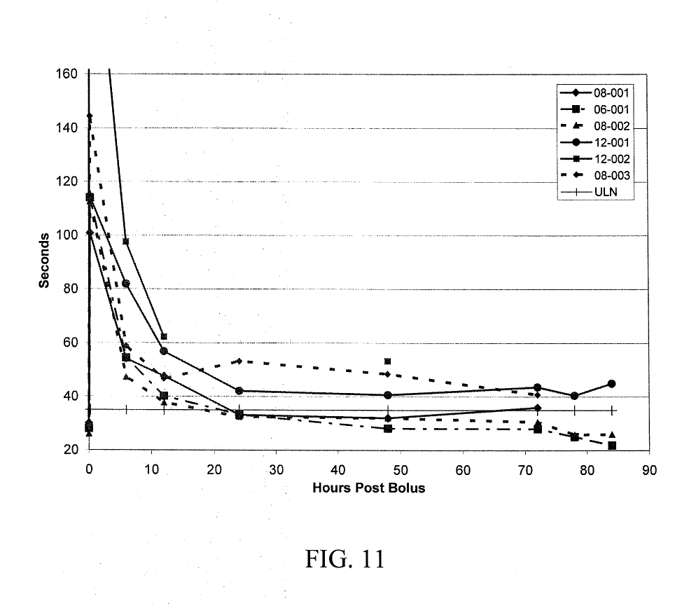

As expected, aPTT averaged about 100 seconds immediately following bolus infusion but then dropped to values approximately 11 seconds above subjects screening baseline at 24 hours, 8-11 seconds above baseline at 48 hours, and to 5-8 seconds above baseline at 72 hours. In no case did aPTT values reach levels of therapeutic anticoagulation. By protocol, two subjects should have had rate reductions for an elevated aPTT on day 2. In one the adjustment was made and in the second it was not. The results for aPTT values during the study in the six subjects are shown in FIG. 11. Platelet counts for the six patients who completed the study are shown below in Table 15, wherein platelet values are provided as thousands/μL blood (mean±SD). O-desulfated heparin did not produce the >50% fall in platelet counts characteristic of heparin-induced thrombocytopenia (HIT), indicating that this heparin analog is safe from producing HIT during use at these doses to treat COPD exacerbations.

TABLE 15

| Subject ID | Entry | Day 2 | Discharge |
|---|---|---|---|
| 08-001 | 379 | 370 | 368 |
| 06-001 | 138 | 163 | 160 |
| 08-002 | 227 | 205 | 210 |
| 12-001 | 261 | 243 | 198 |
| 12-002 | 143 | | 124 |
| 08-003 | 334 | 271 | 275 |
| Mean ± SD | 247 ± 98 | 250 ± 78 | 223 ± 88 |

Hospital admission and discharge times are shown in Table 16 for the six subjects completing the study. Subjects were hospitalized for times ranging from 74.5 to 119 hours, with a mean of 99±17 hours, or 4.1±0.7 days. This duration of hospitalization is almost a full two days less than the average length of hospitalization for the shortest time available in the literature, shown in Table 1, which ranges from 5.9 days to 12 days, and indicates that 2-O, 3-O desulfated heparin infusion hastens the time to improvement of the COPD exacerbation when added to conventional standard of care therapy for these patients.

TABLE 16

| | | | | Hours |
|---|---|---|---|---|
| Subject | Admission Date | Start of ODSH | Discharge | Hospi- |
| ID | and Time | Bolus | Date and Time | talized |
| 08-001 | Jul. 04, 2007 | Jul. 05, 2007 | Jul. 08, 2007 | 110 |
| | 00:11 hours | 21:25 hours | 14:00 hours | |
| 06-001 | Jul. 13, 2007 | Jul. 13, 2007 | Jul. 19, 2007 | 141 |
| | 17:52 hours | 22:24 hours | 14:57 hours | |
| 08-002 | Jul. 24, 2007 | Jul. 24, 2007 | Jul. 29, 2007 | 119 |
| | 16:07 hours | 21:03 hours | 15:05 hours | |
| 12-001 | Aug. 14, 2007 | Aug. 14, 2007 | Aug. 18, 2007 | 97.5 |
| | 12:34 hours | 18:35 hours | 14:13 hours | |
| 12-002 | Aug. 24, 2007 | Aug. 24, 2007 | Aug. 28, 2007 | 102 |
| | 9:04 hours | 22:55 hours | 15:16 hours | |
| 08-003 | Sep. 10, 2007 | Sep. 10, 2007 | Sep. 13, 2007 | 74.5 |
| | 09:30 hours | 17:16 hours | 12:02 hours | |

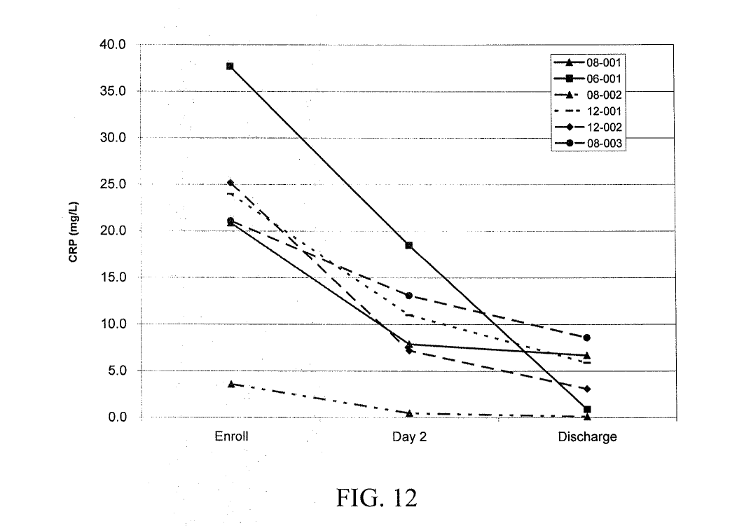

The mechanism by which 2-O, 3-O desulfated heparin therapy shortens the duration of hospitalization required is through decreasing lung inflammation. This is demonstrated in FIG. 12, which illustrates that 2-O, 3-O desulfated heparin infusion dramatically reduces lung and systemic inflammation measured by plasma C-reactive protein (CRP). Normally, even with corticosteroid therapy, CRP falls only by about 50% during the course of hospitalization for COPD exacerbations. The literature indicates that during hospitalization for COPD exacerbations, CRP normally falls from an average of 10.9 to 5.3 mg/L from onset of exacerbation to day 7 of therapy (see table 2 in Perera W R, et al., Eur Respir J 29:527-534, 2007).

By contrast, in the present study, the six subjects experienced a fall in CRP from 22.1±11.0 mg/L on hospital admission to 4.2±3.4 mg/L at hospital discharge, or a decrease of 81% from onset of acute exacerbation. This indicates that 2-O, 3-O desulfated heparin has great utility as a safe therapy to speed improvement of subjects suffering a COPD exacerbation, decreasing the time when they can enjoy sufficient improvement in lung and systemic inflammation and increase in health and well-being to allow discharge to the home environment. This therapy will provide not only an improvement in the health of the individual patient suffering a COPD exacerbation, but will also decrease the overall cost of care for patients requiring hospitalization because of exacerbations of their COPD condition.

Many modifications and other embodiments of the inventions set forth herein will come to mind to one skilled in the art to which these inventions pertain having the benefit of the teachings presented in the foregoing descriptions. Therefore, it is to be understood that the inventions are not to be limited to the specific embodiments disclosed and that modifications and other embodiments are intended to be included within the scope of the appended claims. Although specific terms are employed herein, they are used in a generic and descriptive sense only and not for purposes of limitation.

That which is claimed:

1. A method of treating a patient suffering from an acute exacerbation of Chronic Obstructive Pulmonary Disease (COPD), the method comprising administering to the patient a pharmaceutical composition comprising an amount of O-desulfated heparin effective to lessen or eliminate the acute exacerbation of COPD.

2. The method of claim 1, wherein the acute exacerbation is indicated by the presence of a symptom selected from the group consisting of increased sputum production, more purulent sputum, change in sputum color, increased coughing, increased wheezing, chest tightness, reduced exercise tolerance, increased fatigue, fluid retention, acute confusion, worsened dyspnea, and combinations thereof, and wherein treatment is effective to lessen or eliminate the symptom.

3. The method of claim 1, wherein the O-desulfated heparin is O-desulfated at least at the 2-O and 3-O positions.

4. The method of claim 1, wherein the O-desulfated heparin is at least partially desulfated at both of the 2-O and 3-O positions.

5. The method of claim 1, wherein the O-desulfated heparin is at least about 90% desulfated, independently, at each of the 2-O and 3-O positions.

6. The method of claim 1, wherein the O-desulfated heparin is 100% desulfated at both of the 2-O and 3-O positions.

7. The method of claim 1, wherein the O-desulfated heparin has a molecular weight in the range of about 100 Da to about 30,000 Da.

8. The method of claim 7, wherein the O-desulfated heparin has a molecular weight in the range of about 8,000 Da to about 12,500 Da.

9. The method of claim 1, further comprising administering one or more additional active agents.

10. The method of claim 9, wherein the one or more additional active agents is selected from the group consisting of bronchodilators, anticholinergics, corticosteroids, antibiotics, methylxanthines, and combinations thereof.

11. The method of claim 1, wherein administration is via a route selected from the group consisting of intravenous administration, subcutaneous administration, inhalation, and combinations thereof.

12. The method of claim 1, comprising administering the composition as a bolus comprising O-desulfated heparin in an amount of about 0.1 mg/kg of patient body weight to about 20 mg/kg of patient body weight.

13. The method of claim 1, wherein administering comprises constantly infusing the composition for a time of about 12 hours to about 168 hours.

14. The method of claim 13, wherein the constantly infused composition comprises O-desulfated heparin in an amount of about 0.05 to about 5 mg per kg of body weight per hour of delivery.

15. The method of claim 1, wherein the acute exacerbation requires the patient to be hospitalized, and treatment is effective to lessen or eliminate the acute exacerbation such that the patient is discharged from hospitalization, and wherein the patient is hospitalized for a total time after the onset of treatment of less than five days.

16. The method of claim 1, wherein the treatment is effective to lessen or eliminate the acute exacerbation by reducing lung inflammation in the patient as evidenced by a reduction in the measured level of plasma C-reactive protein (CRP), and wherein CRP is reduced by at least about 60% in a time of less than 120 hours after first administration of the composition.

17. A method of reducing average hospitalization time for a patient suffering from an acute exacerbation of COPD, the method comprising administering to the patient a pharmaceutical composition comprising a treatment effective amount of O-desulfated heparin, wherein average hospitalization time is measured as the time from the onset of treatment in the hospital to the time the acute exacerbation is sufficiently lessened or eliminated such that the patient is discharged from the hospital, and wherein the average hospitalization time for the patient is less than the average hospitalization time for a patient suffering from an acute exacerbation of COPD that is not treated with the O-desulfated heparin.

18. The method of claim 17, wherein the average hospitalization time is reduced by at least about 20%.

19. The method of claim 17, wherein the average hospitalization time is reduced by at least one day.

20. The method of claim 17, wherein the average hospitalization time is reduced by at least two days.

21. The method of claim 17, wherein the average hospitalization time is reduced such that the average hospitalization time for the patient is less than five days.

22. The method of claim 17, wherein the acute exacerbation is indicated by the presence of a symptom selected from the group consisting of increased sputum production, more purulent sputum, change in sputum color, increased coughing, increased wheezing, chest tightness, reduced exercise tolerance, increased fatigue, fluid retention, acute confusion, worsened dyspnea, and combinations thereof, and wherein the time the acute exacerbation is sufficiently lessened or eliminated such that the patient is discharged from the hospital is determined by the symptom being lessened or eliminated.

23. The method of claim 17, wherein the O-desulfated heparin is O-desulfated at least at the 2-O and 3-O positions.

24. The method of claim 17, wherein the O-desulfated heparin is at least partially desulfated at both of the 2-O and 3-O positions.

25. The method of claim 17, wherein the O-desulfated heparin is at least about 90% desulfated, independently, at each of the 2-O and 3-O positions.

26. The method of claim 17, wherein the O-desulfated heparin is 100% desulfated at both of the 2-O and 3-O positions.

27. The method of claim 17, wherein the O-desulfated heparin has a molecular weight in the range of about 100 Da to about 30,000 Da.

28. The method of claim 27, wherein the O-desulfated heparin has a molecular weight in the range of about 8,000 Da to about 12,500 Da.

29. The method of claim 17, wherein the time the acute exacerbation is sufficiently lessened or eliminated such that the patient is discharged from the hospital is determined using the Global Initiative for Chronic Obstructive Lung Disease (GOLD) recommended criteria for hospital discharge.

30. The method of claim 29, wherein the time of discharge is established when at least one of the following criteria is met:
a) Inhaled β₂-agonist therapy is required no more frequently than every 4 hours;
b) The patient, if previously ambulatory, is able to walk across the room;
c) The patient is able to eat and sleep without frequent awakening by dyspnea;
d) The patient has been clinically stable for 12-24 hours;
e) The patient's arterial blood gases have been stable for 12-24 hours; and
f) The patient, family, and physician are confident the patient can manage successfully at home.

31. The method of claim 30, wherein the time of discharge is established when at least two of the criteria are met.

32. The method of claim 30, wherein the time of discharge is established when at least three of the criteria are met.

33. A method for reducing lung inflammation in a patient suffering from an acute exacerbation of COPD, the method comprising administering to the patient a pharmaceutical composition comprising an amount of O-desulfated heparin effective to reduce the lung inflammation, the reduced inflammation being indicated as a decrease in the measured level of plasma C-reactive protein (CRP) of the patient.

34. The method of claim 33, wherein the measured level of plasma CRP is decreased by at least about 60%.

35. The method of claim 34, wherein the measured level of plasma CRP is decreased by at least about 70%.

36. The method of claim 34, wherein the decrease is achieved within a time of less than 168 hours after the onset of treatment.

37. The method of claim 36, wherein the decrease is achieved within a time of less than 120 hours after the onset of treatment.

38. The method of claim 36, wherein the decrease is achieved within a time of less than 72 hours after the onset of treatment.

39. The method of claim 33, wherein the O-desulfated heparin is O-desulfated at least at the 2-O and 3-O positions.

40. The method of claim 33, wherein the O-desulfated heparin is at least partially desulfated at both of the 2-O and 3-O positions.

41. The method of claim 33, wherein the O-desulfated heparin is at least about 90% desulfated, independently, at each of the 2-O and 3-O positions.

42. The method of claim 33, wherein the O-desulfated heparin is 100% desulfated at both of the 2-O and 3-O positions.

43. The method of claim 33, wherein the O-desulfated heparin has a molecular weight in the range of about 100 Da to about 30,000 Da.

44. The method of claim 43, wherein the O-desulfated heparin has a molecular weight in the range of about 8,000 Da to about 12,500 Da.

45. A method of preventing an acute exacerbation of COPD in a patient suffering from COPD, the method comprising administering to the patient, prior to onset in the patient of a symptom indicating an acute exacerbation of COPD, a pharmaceutical composition comprising an amount of O-desulfated heparin effective to prevent onset of the symptom indicating an acute exacerbation of COPD.

46. The method of claim 45, wherein symptom is selected from the group consisting of increased sputum production, more purulent sputum, change in sputum color, increased coughing, increased wheezing, chest tightness, reduced exercise tolerance, increased fatigue, fluid retention, acute confusion, worsened dyspnea, and combinations thereof.
